SEQUENCE LISTING (7 sequences)

SEQ ID NO: 1 - DNA, 1254 bp, Oryza sativa
atggagatggccagtggaggaggcgccgccgccgccgccggcggcggagtaggcggcagcggcggcggtggtggtggaggggacgagcaccgccagctgcacggtctcaagttcggcaagaagatctacttcgaggacgccgccgcggcagcaggcggcggcggcactggcagtggcagtggcagcgcgagcgccgcgccgccgtcctcgtcttccaaggcggcgggtggtggacgcggcggagggggcaagaacaaggggaagggcgtggccgcggcggcgccaccgccgccgccgccgccgccgcggtgccaggtggaggggtgcggcgcggatctgagcgggatcaagaactactactgccgccacaaggtgtgcttcatgcattccaaggctccccgcgtcgtcgtcgccggcctcgagcagcgcttctgccagcagtgcagcaggttccacctgctgcctgaatttgaccaaggaaaacgcagctgccgcagacgccttgcaggtcataatgagcgccggaggaggccgcaaacccctttggcatcacgctacggtcgactagctgcatctgttggtgagcatcgcaggttcagaagctttacgttggatttctcctacccaagggttccaagcagcgtaaggaatgcatggccagcaattcaaccaggcgatcggatctccggtggtatccagtggcacaggaacgtagctcctcatggtcactctagtgcagtggcgggatatggtgccaacacatacagcggccaaggtagctcttcttcagggccaccggtgttcgctggcccaaatctccctccaggtggatgtctcgcaggggtcggtgccgccaccgactcgagctgtgctctctctcttctgtcaacccagccatgggatactactacccacagtgccgctgccagccacaaccaggctgcagccatgtccactaccaccagctttgatggcaatcctgtggcaccctccgccatggcgggtagctacatggcaccaagcccctggacaggttctcggggccatgagggtggtggtcggagcgtggcgcaccagctaccacatgaagtctcacttgatgaggtgcaccctggtcctagccatcatgcccacttctccggtgagcttgagcttgctctgcaggggaacggtccagccccagcaccacgcatcgatcctgggtccggcagcaccttcgaccaaaccagcaacacgatggattggtctctgtag

SEQ ID NO: 2 - protein, 417 aa, Oryza sativa
MEMASGGGAAAAAGGGVGGSGGGGGGGDEHRQLHGLKFGKKIYFEDAAAAAGGGGTGSGSGSASAAPPSSSSKAAGGGRGGGGKNKGKGVAAAAPPPPPPPPRCQVEGCGADLSGIKNYYCRHKVCFMHSKAPRVVVAGLEQRFCQQCSRFHLLPEFDQGKRSCRRRLAGHNERRRRPQTPLASRYGRLAASVGEHRRFRSFTLDFSYPRVPSSVRNAWPAIQPGDRISGGIQWHRNVAPHGHSSAVAGYGANTYSGQGSSSSGPPVFAGPNLPPGGCLAGVGAATDSSCALSLLSTQPWDTTTHSAAASHNQAAAMSTTTSFDGNPVAPSAMAGSYMAPSPWTGSRGHEGGGRSVAHQLPHEVSLDEVHPGPSHHAHFSGELELALQGNGPAPAPRIDPGSGSTFDQTSNTMDWSL

SEQ ID NO: 3 - DNA, 2593 bp, Oryza sativa
tccggcggtaagcacggttcccacgtggtaaccgcggtaattgtaaaaaaccatgcaaaatttatcaaaaatttaaattatttttaaaatttatttaaatttaaggaggttaccgcggtatttatattaccgtacccccgcggtaagcccggtaaccacgcggttaccggcggtaagggaaaccctgccacatactactctaccctgccacatactactctacgccaaaattttagtaacaattatagcaaagaaaaatctagtagactctccaactggatggccatgccatctagctggccaaatccctcccctcgatggtcaaacttggccagccaaaaatcacttgtcaacggtgtgcctcacgtgctattcctcccctcttctctctgttgtgctgatggataagaggcgtcgacggtgaccgttgggttaagctaggacggtgcagtgctggacgtgaggaggcggttgtgccggagcctaccccttggcactggcggtgttgggagggggggttgggcggtgggagatggcaaagcgacactgggttgcgtgagaggcaaacggtgctaggttgtcggagtccacgaattagagagtgggccaattcgtacagcggcgcgcgcggagggtggccttctcgaggtgaagtcttgactcctatgctgaggtcttggatcagccataaagctctggtcgacgtcatcgccaaaacccaccccttcccctccatcctctccgccgccacgccccctagataactattcacccttcgttcggagcccaccactgtctctactctctaccatcatagcgcctgagaatagttatgagagggcaaggacggtagggtgggtgggaggagaaagacgaaggagggagaaagaaaaagtaaagagagactgacacacgggacccactatccgattggattgtacagtgaagatgttgttggagtgaaagtcaaatttgacttttttttaagcttgtcaaatggatattttgagatgctgtcggatatactctaagctaccacatggtgtggccatacatgcgcgagtaccccatggttaacacagtaagctcatacggtaaccattttagttattttttaccgtattaacctcgtgccgtaaccatcttagttttcaaatcataccattttttctcgctgaccacggtagccatgatatcattgggtagaggtaacttctaccaaagcaatagtgaatgtgtaccatggagagaagccaaagccgtgcacgccggagaaaattgatgttatgccatcgtcggaaagggatttcgctaaaatatcataacacatttaggattcaataaaatggtactctcaaataggatcaacacgccgcaatgacatttctttccttcttaatttaatgatttttcatgtttgctttggcaaagctaaccaaatgcccttcttcctcttgatccctttttttgtctcattgatgagagctcgacgaagtagtagcaccaccatggctagggaagaggtcgtaaatcaaagatatcatgcccgcgcgctcgtcgatagccgcaacaggcgtcttctactcttcaacgtggcggggaggtcctccaagatgaacctgatgcctttaccagtggtacaaccctcgcacaagagcgaacgaagatcgccggccttggtctccactacctccctcacacttcgtgcaaggacaaagagggacacgaactggaggttcttgaggcagttgtgcctgccctgtccatcgccgccactcgcaaccacaccaagatgtgaagcatccgttgtctggacctccatggccacgccaacgcaaacacccttgtccaatacttttcaatagtgtcgccacatggtcgaagctccatcaccgcctccgatctaagcctcaccacccttatcccaatgttcatctcaacatcctttcctcttctacttcccctacaagatcgagtaaaagtctcagcaaagattcacaatataatttctcttttatttctcctacgagatcgagtagaggtctacgtaaagatttaataagtgcccattatttaatagtaacattttattgaatctcgtttagagcaggtacaatagcaggctacaagtcagctataagcacatgtggaagagaaaaaagaggagagagaagcaaagcaggctacaaatttgtagccagctgcagcacagactccaaaacgttatatgtgtacgagaggcgggaccatatattaatggtgtagtatatgtttataagtaactattgtatgaataaactattagattggctagctataatagtaagctccaaatcatctatagccaatataatagccaattcatacaatagttgcttactatactattaatacctggtcccaccaatcacacacacgttaggtcttggagtctgtgctgcagctggctacagatttgtaacccgctgctcttctctcttcctttatttttttaaagagtaaatttcataaaactacagatactttgcatgatctatcacaaaactacagattttagaactagtttcacaaaactacagatttaatgtgttca

SEQ ID NO: 4 - DNA, 2591 bp, Oryza stiva
ccggcggtaagcacggttcccacgtggtaaccgcggtaattgtaaaaaaccatgcaaaatttatcaaaaatttaaattatttttaaaatttatttaaatttaaggaggttaccgcggtatttatattaccgtacccccgcggtaagcccggtaaccacgcggttaccggcggtaagggaaaccctgccacatactactctaccctgccacatactactctacgccaaaattttagtaacaattatagcaaagaaaaatctagtagactctccaactggatggccatgccatctagctggccaaatccctcccctcgatggtcaaacttggccagccaaaaatcacttgtcaacggtgtgcctcacgtgctattcctcccctcttctctctgttgtgctgatggataagaggcgtcgacggtgaccgttgggttaagctaggacggtgcagtgctggacgtgaggaggcggttgtgccggagcctaccccttggcactggcggtgttgggagggggggttgggcggtgggagatggcaaagcgacactgggttgcgtgagaggcaaacggtgctaggttgtcggagtccacgaattagagagtgggccaattcgtacagcggcgcgcgcggagggtggccttctcgaggtgaagtcttgactcctatgctgaggtcttggatcagccataaagctctggtcgacgtcatcgccaaaacccaccccttcccctccatcctctccgccgccacgccccctagataactattcacccttcgttcggagcccaccactgtctctactctctaccatcatagcgcctgagaatagttatgagagggcaaggacggtagggtgggtgggaggagaaagacgaaggagggagaaagaaaaagtaaagagagactgacacacgggacccactatccgattggattgtacagtgaagatgttgttggagtgaaagtcaaatttgacttttttttaagcttgtcaaatggatattttgagatgctgtcggatatactctaagctaccacatggtgtggccatacatgcgcgagtaccccatggttaacacagtaagctcatacggtaaccattttagttattttttaccgtattaacctcgtgccgtaaccatcttagttttcaaatcataccattttttctcgctgaccacggtagccatgatatcattgggtagaggtaacttctaccaaagcaatagtgaatgtgtaccatggagagaagccaaagccgtgcacgccggagaaaattgatgttatgccatcgtcggaaagggatttcgctaaaatatcataacacatttaggattcaataaaatggtactctcaaataggatcaacacgccgcaatgacatttctttccttcttaatttaatgatttttcatgtttgctttggcaaagctaaccaaatgcccttcttcctcttgatccctttttttgtctcattgatgagagctcgacgaagtagtagcaccaccatggctagggaagaggtcgtaaatcaaagatatcatgcccgcgcgctcgtcgatagccgcaacaggcgtcttctactcttcaacgtggcggggaggtcctccaagatgaacctgatgcctttaccagtggtacaaccctcgcacaagagcgaacgaagatcgccggccttggtctccactacctccctcacacttcgtgcaaggacaaagagggacacgaactggaggttcttgaggcagttgtgcctgccctgtccatcgccgccactcgcaaccacaccaagatgtgaagcatccgttgtctggacctccatggccacgccaacgcaaacacccttgtccaatacttttcaatagtgtcgccacatggtcgaagctccatcaccgcctccgatctaagcctcaccacccttatcccaatgttcatctcaacatcctttcctcttctacttcccctacaagatcgagtaaaagtctcagcaaagattcacaatataatttctcttttatttctcctacgagatcgagtagaggtctacgtaaagatttaataagtgcccattatttaatagtaacattttattgaatctcgtttagagcaggtacaatagcaggctacaagtcagctataagcacatgtggaagagaaaaaagaggagagagaagcaaagcaggctacaaatttgtagccagctgcagcacagactccaaaacgttatatgtgtacgagaggcgggaccatatattaatggtgtagtatatgtttataagtaactattgtatgaataaactattagattggctagctataatagtaagctccaaatcatctatagccaatataatagccaattcatacaatagttgcttactatactattaatacctggtcccaccaatcacacacacgttaggtcttggagtctgtgctgcagctggctacagatttgtaacccgctgctcttctctcttcctttatttttttaaagagtaaatttcataaaactacagatactttgcatgatctatcacaaaactacagattttagaactagtttcacaaaactacagatttaatgtgttc

SEQ ID NO: 5 - DNA, 3827 bp, Oryza sativa
  variation (1)..(1)
  variation (29)..(29)
  variation (2621)..(2621)
  variation (3474)..(3474)
  variation (3827)..(3827)
taaaccgtcgggatcagggttactacgctccggcggtaagcacggttcccacgtggtaaccgcggtaattgtaaaaaaccatgcaaaatttatcaaaaatttaaattatttttaaaatttatttaaatttaaggaggttaccgcggtatttatattaccgtacccccgcggtaagcccggtaaccacgcggttaccggcggtaagggaaaccctgccacatactactctaccctgccacatactactctacgccaaaattttagtaacaattatagcaaagaaaaatctagtagactctccaactggatggccatgccatctagctggccaaatccctcccctcgatggtcaaacttggccagccaaaaatcacttgtcaacggtgtgcctcacgtgctattcctcccctcttctctctgttgtgctgatggataagaggcgtcgacggtgaccgttgggttaagctaggacggtgcagtgctggacgtgaggaggcggttgtgccggagcctaccccttggcactggcggtgttgggagggggggttgggcggtgggagatggcaaagcgacactgggttgcgtgagaggcaaacggtgctaggttgtcggagtccacgaattagagagtgggccaattcgtacagcggcgcgcgcggagggtggccttctcgaggtgaagtcttgactcctatgctgaggtcttggatcagccataaagctctggtcgacgtcatcgccaaaacccaccccttcccctccatcctctccgccgccacgccccctagataactattcacccttcgttcggagcccaccactgtctctactctctaccatcatagcgcctgagaatagttatgagagggcaaggacggtagggtgggtgggaggagaaagacgaaggagggagaaagaaaaagtaaagagagactgacacacgggacccactatccgattggattgtacagtgaagatgttgttggagtgaaagtcaaatttgacttttttttaagcttgtcaaatggatattttgagatgctgtcggatatactctaagctaccacatggtgtggccatacatgcgcgagtaccccatggttaacacagtaagctcatacggtaaccattttagttattttttaccgtattaacctcgtgccgtaaccatcttagttttcaaatcataccattttttctcgctgaccacggtagccatgatatcattgggtagaggtaacttctaccaaagcaatagtgaatgtgtaccatggagagaagccaaagccgtgcacgccggagaaaattgatgttatgccatcgtcggaaagggatttcgctaaaatatcataacacatttaggattcaataaaatggtactctcaaataggatcaacacgccgcaatgacatttctttccttcttaatttaatgatttttcatgtttgctttggcaaagctaaccaaatgcccttcttcctcttgatccctttttttgtctcattgatgagagctcgacgaagtagtagcaccaccatggctagggaagaggtcgtaaatcaaagatatcatgcccgcgcgctcgtcgatagccgcaacaggcgtcttctactcttcaacgtggcggggaggtcctccaagatgaacctgatgcctttaccagtggtacaaccctcgcacaagagcgaacgaagatcgccggccttggtctccactacctccctcacacttcgtgcaaggacaaagagggacacgaactggaggttcttgaggcagttgtgcctgccctgtccatcgccgccactcgcaaccacaccaagatgtgaagcatccgttgtctggacctccatggccacgccaacgcaaacacccttgtccaatacttttcaatagtgtcgccacatggtcgaagctccatcaccgcctccgatctaagcctcaccacccttatcccaatgttcatctcaacatcctttcctcttctacttcccctacaagatcgagtaaaagtctcagcaaagattcacaatataatttctcttttatttctcctacgagatcgagtagaggtctacgtaaagatttaataagtgcccattatttaatagtaacattttattgaatctcgtttagagcaggtacaatagcaggctacaagtcagctataagcacatgtggaagagaaaaaagaggagagagaagcaaagcaggctacaaatttgtagccagctgcagcacagactccaaaacgttatatgtgtacgagaggcgggaccatatattaatggtgtagtatatgtttataagtaactattgtatgaataaactattagattggctagctataatagtaagctccaaatcatctatagccaatataatagccaattcatacaatagttgcttactatactattaatacctggtcccaccaatcacacacacgttaggtcttggagtctgtgctgcagctggctacagatttgtaacccgctgctcttctctcttcctttatttttttaaagagtaaatttcataaaactacagatactttgcatgatctatcacaaaactacagattttagaactagtttcacaaaactacagatttaatgtgttcatttatcacaaaactacatgtactttgaacactatatcacaaaactacagatttaagaacttgtttcacaaaactacaggtttagtatcttcatttttcacaaaactacaaatttaatatcttcatttatcacaaaactacagattcagtgtctccattctcataaaactacacgttttaataatgctaaaactattattaaaacgtatagttttatgagaatggagacactgaatctgtagttttgtgataaatgaagacactaaatgtgtagcattgttataaacgaatacactaaatctatatttttgtgaaacaagttcttaaatatgtagttttgtcatagattgtacaaagtacctgtagttttgtgataaaacgaagacactaaatctgtagttttgtgataaattgtacaaaaatacctgtagttttgtgaaatttactcttttttaaaatatgtttatagctggcttaggccatttaaagtgcaaggacgtggcgtgtggcaatgtagagccacgtaggcaagtcgcttgcgtggaggagagaggggagtggggaccgttcccaacccagcttcgtgtgaccaagtttggccacacgggccaaacgaacctcagcaacttttgtcagaaagaaaagaacctccgcgagaaacaagaaagcgagagagggagagaaaggaggctcgtcggagtaggggcgctcggggtatggggctcggcggaggctcgctggagtaggggccgccactgcgtggggctcgccggagtaggggcgctcggggagcctcccgagatccgccgctcagcggcgccgccgtcttccgggcagagctctcgaagctcgctctcctcgcgcgccggtggcgttggcggggcccgcgtgtggctacgcagctccggtgctgcgcctccaccgtcgacgacagcgccgcttggcgctgccgccgtcttccgccgcgccgctggacgccgccagatctgctgctcgtcgccgcgtgggccgctccacccggttggaggaggagaggcggcgccgcgcttgggctgccccaccgccgagctctgccgcgccgttcgccggtgctgccgagctccgccgcgcctgccggagcacgctgccatggccgccctggagaagacacgagagaattaggtggagggtgggggaagggtgagattttttatattatctatgggtt

SEQ ID NO: 6 - DNA, 8028 bp, Oryza sativa
taaaccgtcgggatcagggttactacgctccggcggtaagcacggttcccacgtggtaaccgcggtaattgtaaaaaaccatgcaaaatttatcaaaaatttaaattatttttaaaatttatttaaatttaaggaggttaccgcggtatttatattaccgtacccccgcggtaagcccggtaaccacgcggttaccggcggtaagggaaaccctgccacatactactctaccctgccacatactactctacgccaaaattttagtaacaattatagcaaagaaaaatctagtagactctccaactggatggccatgccatctagctggccaaatccctcccctcgatggtcaaacttggccagccaaaaatcacttgtcaacggtgtgcctcacgtgctattcctcccctcttctctctgttgtgctgatggataagaggcgtcgacggtgaccgttgggttaagctaggacggtgcagtgctggacgtgaggaggcggttgtgccggagcctaccccttggcactggcggtgttgggagggggggttgggcggtgggagatggcaaagcgacactgggttgcgtgagaggcaaacggtgctaggttgtcggagtccacgaattagagagtgggccaattcgtacagcggcgcgcgcggagggtggccttctcgaggtgaagtcttgactcctatgctgaggtcttggatcagccataaagctctggtcgacgtcatcgccaaaacccaccccttcccctccatcctctccgccgccacgccccctagataactattcacccttcgttcggagcccaccactgtctctactctctaccatcatagcgcctgagaatagttatgagagggcaaggacggtagggtgggtgggaggagaaagacgaaggagggagaaagaaaaagtaaagagagactgacacacgggacccactatccgattggattgtacagtgaagatgttgttggagtgaaagtcaaatttgacttttttttaagcttgtcaaatggatattttgagatgctgtcggatatactctaagctaccacatggtgtggccatacatgcgcgagtaccccatggttaacacagtaagctcatacggtaaccattttagttattttttaccgtattaacctcgtgccgtaaccatcttagttttcaaatcataccattttttctcgctgaccacggtagccatgatatcattgggtagaggtaacttctaccaaagcaatagtgaatgtgtaccatggagagaagccaaagccgtgcacgccggagaaaattgatgttatgccatcgtcggaaagggatttcgctaaaatatcataacacatttaggattcaataaaatggtactctcaaataggatcaacacgccgcaatgacatttctttccttcttaatttaatgatttttcatgtttgctttggcaaagctaaccaaatgcccttcttcctcttgatccctttttttgtctcattgatgagagctcgacgaagtagtagcaccaccatggctagggaagaggtcgtaaatcaaagatatcatgcccgcgcgctcgtcgatagccgcaacaggcgtcttctactcttcaacgtggcggggaggtcctccaagatgaacctgatgcctttaccagtggtacaaccctcgcacaagagcgaacgaagatcgccggccttggtctccactacctccctcacacttcgtgcaaggacaaagagggacacgaactggaggttcttgaggcagttgtgcctgccctgtccatcgccgccactcgcaaccacaccaagatgtgaagcatccgttgtctggacctccatggccacgccaacgcaaacacccttgtccaatacttttcaatagtgtcgccacatggtcgaagctccatcaccgcctccgatctaagcctcaccacccttatcccaatgttcatctcaacatcctttcctcttctacttcccctacaagatcgagtaaaagtctcagcaaagattcacaatataatttctcttttatttctcctacgagatcgagtagaggtctacgtaaagatttaataagtgcccattatttaatagtaacattttattgaatctcgtttagagcaggtacaatagcaggctacaagtcagctataagcacatgtggaagagaaaaaagaggagagagaagcaaagcaggctacaaatttgtagccagctgcagcacagactccaaaacgttatatgtgtacgagaggcgggaccatatattaatggtgtagtatatgtttataagtaactattgtatgaataaactattagattggctagctataatagtaagctccaaatcatctatagccaatataatagccaattcatacaatagttgcttactatactattaatacctggtcccaccaatcacacacacgttaggtcttggagtctgtgctgcagctggctacagatttgtaacccgctgctcttctctcttcctttatttttttaaagagtaaatttcataaaactacagatactttgcatgatctatcacaaaactacagattttagaactagtttcacaaaactacagatttaatgtgttcatttatcacaaaactacatgtactttgaacactatatcacaaaactacagatttaagaacttgtttcacaaaactacaggtttagtatcttcatttttcacaaaactacaaatttaatatcttcatttatcacaaaactacagattcagtgtctccattctcataaaactacacgttttaataatgctaaaactattattaaaacgtatagttttatgagaatggagacactgaatctgtagttttgtgataaatgaagacactaaatgtgtagcattgttataaacgaatacactaaatctatatttttgtgaaacaagttcttaaatatgtagttttgtcatagattgtacaaagtacctgtagttttgtgataaaacgaagacactaaatctgtagttttgtgataaattgtacaaaaatacctgtagttttgtgaaatttactcttttttaaaatatgtttatagctggcttaggccatttaaagtgcaaggacgtggcgtgtggcaatgtagagccacgtaggcaagtcgcttgcgtggaggagagaggggagtggggaccgttcccaacccagcttcgtgtgaccaagtttggccacacgggccaaacgaacctcagcaacttttgtcagaaagaaaagaacctccgcgagaaacaagaaagcgagagagggagagaaaggaggctcgtcggagtaggggcgctcggggtatggggctcggcggaggctcgctggagtaggggccgccactgcgtggggctcgccggagtaggggcgctcggggagcctcccgagatccgccgctcagcggcgccgccgtcttccgggcagagctctcgaagctcgctctcctcgcgcgccggtggcgttggcggggcccgcgtgtggctacgcagctccggtgctgcgcctccaccgtcgacgacagcgccgcttggcgctgccgccgtcttccgccgcgccgctggacgccgccagatctgctgctcgtcgccgcgtgggccgctccacccggttggaggaggagaggcggcgccgcgcttgggctgccccaccgccgagctctgccgcgccgttcgccggtgctgccgagctccgccgcgcctgccggagcacgctgccatggccgccctggagaagacacgagagaattaggtggagggtgggggaagggtgagattttttatattatctatgggttccattataaattttctaaaccacacttatactgtgggtgcagtgtcatttagagttcccaaaccacctatgttgcagctgtggtataacaatttgctaggacgcattgctactgcccttgtaccctgctataagaagataaccaatgacatctccactcgattttctcggcgcgcgtgtgagggtgtgaggataatttttattttaagtggtttttaagggcggagagagagagagagagagggcaccgcactacttctacttgtgtgtgtgtcgctcgctgggcttcgccacctttccgtctctttcctctctcttctctctccccctctcctggaggagagagaggagaagaggagggggggccgcgccaagagccacgcgcgctacagtctccttcccacccgcgaccgcgagcaatggagatggccagtggaggaggcgccgccgccgccgccggcggcggagtaggcggcagcggcggcggtggtggtggaggggacgagcaccgccagctgcacggtctcaagttcggcaagaagatctacttcgaggacgccgccgcggcagcaggcggcggcggcactggcagtggcagtggcagcgcgagcgccgcgccgccgtcctcgtcttccaaggcggcgggtggtggacgcggcggagggggcaagaacaaggggaagggcgtggccgcggcggcgccaccgccgccgccgccgccgccgcggtgccaggtggaggggtgcggcgcggatctgagcgggatcaagaactactactgccgccacaaggtgtgcttcatgcattccaaggctccccgcgtcgtcgtcgccggcctcgagcagcgcttctgccagcagtgcagcaggtcactctctcactcacctcgccattgctgatgtcaccactgcttttgctttgctttgcttgctctccctcctctttcacctatctctcttgtttatttgcttcttgttcttgtttagtgctagtacatgtgttgttattgttgtgccgttttgtcttttgggttattgtgttgttgttactactcgttttactataggtttttaaggtttatgagcacggccaccacattagatgcactgtcaagtggtgtgtgtgggacctttcctgctaaaacaagctgatttcaactctctgaaacttcctgcatttcatctatttttatctttgattgtgttgggagtactacactagtagtgttaatattttgactggtgcttatgagatttttaagttggtaggttgatgaggaaaatactcctttatatggttgagtgatgtgacttgcctgtctgcctgcctgcctgccgctttgcataagattcctctgtgttagtaagagccactgtttatttgtactggtgcttactctacttagttaattagccattagctataaaattccgttgatgttgcaagcttagcaatggccacggtaagaatgggagagagaagttggctaaagctgttgctttgtagtttgtactatatatgtgtctttgtgttgcaagatatgcaactcctactatgctgtgacttgagctcaaggttttcagttatctatagatccttactactactgagcatactaccacttctgtatggtagcatatggtagcatagtccaagttccaacgcctcgccagttgttcataatctatactaccacttctgtgcatttgttacttttatttaatagtttgtctcattagctgacaagcatatgcctgttttgatatctgcccctcttgtaatagtctatggatagcttggactgtttgatgctttaattttttactagcaacacttagggcccctttgaaatggaggattagcaaaggaattttggaggattcattttcctaaggattttttcctatagagccctttgattcatagaaagaggataggaaaacttccgtaggattgcattcctatgatcaattccataggaaaataagcaagaggttagacctcttgtgaaactttcctttgttgagtgtatcttgtggtataatcaaagggctcttctctccatttcatgtgttttcaattcctgtaggattggaaaaacatacaacttcaattcctacgtttttcctattcctatgtttttcctatcctgcgtttcaaaggggcccttaaggatgaagggaagtaagagaaacatactagagaatatgtagtagtatttctacattccatatttgtagcactagcccacaaatatctttgccttgtacttacttcataccagttccccccttttcagagcaaaccaacaatttctgttgccttatatatctagtgtcttcgtactaatatatctgttccaaaatgtacctgtccaaattcatagctagaaatagctttatttaggacggaagtaataactgttgttagagacttggttcagacttttggttatgttgaggctactatcatttcctttacgggccaaattactacaaatgagaattcataaaaatgtcaagattttatgattgttgtagctttatttaggacggaggtagtaattgttgttagagacttggttcagacttttggttacgttgaagctactatcatttcctttatggtcaaattactaacaatgagtattcataaaaatgtcaagattttataattgagctgtgccagtgctaagtgtgtcactatctgatgccataatgcatcattataaaagccagatggaccattagcttttatgtgtaggacacctgccgtccaattagatggataaccatctagtgtttgtgtactgttattttaagcccgacatctcacaactccatgaatgattacagtcttcctttcacatggtgtccttttgttgtgttaggaatagcattttttatttatgggtgtaattatgaaaggcactaggagagttgctgctttatcttgatgggatttgtagtaataccatctttaggatgacaagaaatcttgttctgagttagcatgggctgccttttgacctgagctacggtttgctatgtttggcttgcatcatgcagatctattaggataataagcatataaaagttgcttgcattgtgcattgcttgttttaccttgattcatgtaggagtaatttgctcgccatgcctcgttttgctttctgagtcaacagccaaatttagatgatgtaccttctgttgcttcaaaaactcagtcactgcacagcagcagtggataggattcagaatcaatctatccatgattctctgttcacataatatgacaggttccacctgctgcctgaatttgaccaaggaaaacgcagctgccgcagacgccttgcaggtcataatgagcgccggaggaggccgcaaacccctttggcatcacgctacggtcgactagctgcatctgttggtggtatcatcagaggctcttgttttctttgcatcttgtgtgtttgttggtaactactggttgcattcgctgatgtgttgtttgttgcgattcttgatccagaagagcatcgcaggttcagaagctttacgttggatttctcctacccaagggttccaagcagcgtaaggaatgcatggccagcaattcaaccaggcgatcggatctccggtggtatccagtggcacaggaacgtagctcctcatggtcactctagtgcagtggcgggatatggtgccaacacatacagcggccaaggtagctcttcttcagggccaccggtgttcgctggcccaaatctccctccaggtggatgtctcgcaggggtcggtgccgccaccgactcgagctgtgctctctctcttctgtcaacccagccatgggatactactacccacagtgccgctgccagccacaaccaggctgcagccatgtccactaccaccagctttgatggcaatcctgtggcaccctccgccatggcgggtagctacatggcaccaagcccctggacaggttctcggggccatgagggtggtggtcggagcgtggcgcaccagctaccacatgaagtctcacttgatgaggtgcaccctggtcctagccatcatgcccacttctccggtgagcttgagcttgctctgcaggggaacggtccagccccagcaccacgcatcgatcctgggtccggcagcaccttcgaccaaaccagcaacacgatggattggtctctg

SEQ ID NO: 7 - DNA, 21489 bp, Oryza sativa
  misc_feature (10776)..(14602)
gcactacgccccgcaagtcgacaacggcacccgtgcgcatccagcttgcgcccaggaacccacctccatcagcagctcccgcagcacctctagtttcgcccgcagctccagcagttcctgcagctccagcaccagctccagctcccgttcccgtgtcagctcctgcatccgctctctccttcgcgcctgcatcagccgtcagggtccccgccagtggcaatggaggttggttgtcagctactcccagtggcagtcgcgacctcagcagtagcagcttaagtagctcagagctgggcagcggcgagacttatctgccaggctccaggagtcgctcagagggacctggtgatgagcaggaggacgccttcgactccaccgaccgcaggagccgttccgagcattaggctcgttttccattagcttatcgactgtctagcttctgcttgttagtttgtgttttttaggttgcttgcttggggtcagtcgacggtcgatatcatgtgcctatgatatggctgtcttaataaggattttgctttctgtttaagtgttttaattcagatcagaacaattgcagtttaaattcctatataataaagcttagttcctgagtatctgtttttctccgtttccccgtctactcttcctccagatggtctacaccaggactggcacccgcacgacaggtgagggcagcaacggcgaagaacgcgctgatggtgtgcatcccaacagtgattccggcaatggtccgccaccacttcccgagaatccgacgctagcccaagtgatggcacatcagactcagatgatggcagccatgatgcagcagatgcagcagcaacaccagcagatgcaccagaggatgctgcagcacgccgaacagcagcaccagcagtttggtcgccctccccctcagtccaagttgccagagtttctacgtgttaggctcgtccgcgcaagccaagaaggagactggaacaaaaccagggtcctgcttcaactgtggcgagcttggccacttcgctgacaaatgcccgaatcccaggcgtgccgggccaaggtttgtccaggctcgtgtcaaccacgcgtctacataagaggcgcaagcagcaccagaggtcgtactgggtacatttcctgttaactcaatccctgcaacagtactttttgattctggtgcaacacattctttcatttcaaagaaatttgtggggatgcatgggttaaggaaagaagagcttaatacaccaatgagggttcataccccgggaaatagttcaaccttggtcagttttagcccttcagtgctaaaagaaatccaaagatcaccatttctagccaacctcattcttcttgaatccaaagacctagatgtcattcttgggatggattagctgaccaagttcaagggagtcattgattgtgcgaatcgcacagtcaccttgaccaacgagaagggagaaaccgtagtgtacgaatcactagtctcaccaaagcaaggggtctccttgaatcagattgaggcggaaatcccagtggtcaccgaggagaagaatccgaggaaattggaagagattcctatagtttgcgagtacccagaagtctttcccgaagacctcactaccatgccacccaagcgagagattgaattccagattgatttggcaccaggaaccgtgccaatttacaagagaccatacaggatggcggccaacgaactagcagaggtcaagaagcaggttgatgagcagctgcagaaagggtacattcgatcaagtacttcaccttggggtgctccggtcatttttgtggagaaaaaggataagacgaagaggatgtgcgttgattatcgcgcactcaatgaggtcacaatcaagaataaatatcctttaccgcggatcgatgatttgtttgatcaactgaagggagctaaggtgttctctaagatagacctgcgatcaggctatcatcaattaagaatccgggaagagcatattccaaagatagctttcaccacttggtacgggctgtacgagtgcacggttatgtctttcggacttaccaacgcacccgcattcttcatgaatctgatgaacaaagtgttcatggaatttctcgataagttcgttgtggtcttcatcgatgacatacttatctattcaaagtctgaaaaagaacatgagcagcatctccgtctggtgctcgagaagttgaaggagcaccagttatacgccaagttcagcaagtgtgatttttggttgacggaagtcaaatttctgggccatgttattacagctcagggtgtggcagtcgacccatcaaacgtggagtcagttaccaaatggaccccaccgaagaccgtgtcgcagattcgaagttttcttggacttgcaggttattactgccggttcatcgagaacttctccaggatcgcccgacccatgactcagttgcttaagaaagatgagaagtttaaatggacagctgagtgcgataagagctttgaggagctcaagaagaaattagtatccgcaccagttttgattctgcccgaccagatgaaagacttccaagtctattgcgacgcatcccgccacgggcttaggtgtgttctaatgcaggagggcagagtggttgcgtatgcctcgcgacagttgcgtccacacgaagggaattacccaacccatgatctggaattggcagcagtggttcatgctttgaagatctggaggcactatctgattggcaaccgctgtgaagtatatacagaccacaaaagtctgaaatatatcttcacccaaccggacctaaatctccgacaacgaagatggttggaactaatcaaagattatgacatgagcattcactaccacccggggaaagccaatgtggtggcagacgcattaagcaggaaaagttactgcactgccctatgcatcgagggcatgtgtgaggaattgcagcaggagtttaagcgtctcaacgtgggaattgttgagcatgggttcgtagccgctctggaagcacggcctacgctcgtcgatcaagttagagccgctcaagtgaatgatcctgaaatagcagagctgaaaaagaacatgagagtcggaaaggctcgggatttccttgaggatgaacatggcacaatctggatgggggaaagattgtgtgtacctgacgacaaggaactgaaggatctgatactcaccgaggctcatcagacacagtattccattcacccaggcagcaccaagatgtaccaagacctcaaagaaaagttttggtgggtcagcatgaggagagaaatagctgaattcgtcgcactctgcgatgtttgccagcgagtgaaggcagagcatcaaaggccagcaggtttactccaacctcttcaaatcccggaatggaaatgggaggatattagaatggattttataacaggactgccccggacatcatcaggtcacgattcaatctgggtagtcgtggatcgactcaccaaggttgctgtcacgcctagaaattcccgaatagaattcaaagcagaatgtgcattaaaatccccgtccaggaccggccggggtacacaaacgacaatgttgacattcagatccacgtcttacaaacatcataaaagtcttacataaatgcagcggaaaaacaaaagataactggggctaagcttggacttgaactgcagcgggaacaccactccacaggcaccctcgacggcacggacgaagcctactcttcggagaacccaccatcgtacacatactcgcactctggggttgggggaaaatagagcaagactgagtactacccactgtactcagcaagtcataccggaataggggtatgatgcaaggaattatcaaaggagagctagagtggttcatttgcataaagcgagcatttataaacagaagtttaaagaagaaaacagtcgtaatatttaatcaatattaatcatccactgtccaacgctataccacgttgcgacaggcccaaccatccacctatactatccaatttcaaaagactaaactagggtgagactaatcacggtgaatctggttgaccgcccataaccgcgggcacggctattcgaatagttttactctgattagaggtgtacaactgtacccacaagacacagccccacgacacgtttccgtgtgaagacatgccaccacgacataccggaaagaggccgtgacaggacccttcgcataaccccctctaaccaagcacaccacacctcaggtttcacccccactcctcgcaaggcagcgggcagtctcctctcatgcctaggtgaatccggaagcgacagaggccgtcgcagggcccgccccgctccatcacgcccacccttgcctggatgcgtcagctagaggaaagttacactacaagcccagccgttgcccacgctggcttgtggtaagtacgataagttcttccagggcatcccgcgaaccggtccttaactgccatgggtgcgactagcaaaaccatgcactcacagcccaccatgtgattcattttaattaaccaacaccaaaacggtggtactaaaccaaccttaccaattgaacctaaagtctatattttagtgagctttccccattgtgtgctagtggaactaagcatggctaagcaattcctagtccaaaatctattcatgttataacccaagctaacaaaggagcatggtatcaaaaatggcatggctgtaacaataggtaaacaccccatagtaacattataaaacaatgcaatatttgaagaaaaacaatagagcatttgcaatataggatcaacatgttcaagtgacaagcatgacttgccttgctctgcagctggaggaacctcggcgactttctcgaagtacaccggagcgtcggaagaaccggaatctaggcgacatacaaagcaaacaataaaaacaggctataagactactgaaacagagaacaaaaccatttttaatggattctacgcatttttcttgatttactgagacttgaatgggcttaaacggagctcggatgaattagttatgaattttagaagattcactgtgtttattactataacaaaaagtccttaaataatttattgtgcaataattcccagggctgacgtcatcaagggggggcgcggcgccgacatgtggggcccaccggtcagtggctcgggagagagggagaaggggcgccggcacgcgggccccactcgacagcggctcaagggggggcggtccaccgtggaccgggaccacgcgggtggtccaccgccggtccacgggaccgacggcccagattgcccctggggctgatcggacgggcggcggcgacccggctcggctcggctcaaggccggccgaccggccacggcgacagcaaccggcggcggaggcggcggcgcaaaggcgacgcgaagacggcggcgctaagcggcggctaggaaggagagggaggggagtagaggggagaggacgcctcaccgagggtggccggcgcggaggaagacgacggcgaacggcgacggcaagccacgggaggacgacggcaatcggcggacgggcttcggggtgcccggagcgcgaggggatgcgaattccggcgacgaatcctgacggaggaagggcggacggggtggagttcgtccttgcgaacccgacggcggcgacggcgcggtgcggcagcgagcctagcggcggctagcggcggccggagcggcgggaaaggcggcggcgggtgggtgcacggcgcgggagcgatggggagcacgagggagttcggcgaaatggggaaaaagagagagggggcgacggcggagcttaaataggggaggggagtcccggacgtggccggtaggggcgagattccaccggccaacgtgggggagtgggagaggagagagagagtcgggattcgaaaatcgaatcccagccatctcgggcgcgggcgcgggcgggagagagaggggagtgggcgcgggaggtgcggcgcatgcgcgggcgttgcaacatgaatagacgttggactaggagttgcttagccatgcttagttcaactagcacacaatggggaaatctcatttaaaatatggactgtaggttcttatgataatgttggtttagtaccaccgctttggtgttggttaattaaaatgaatcacatggtgggctgtgggtgcatggttttgcttgtcgcacccatagcaattaaggaccggttcgcgggaaaccctggaagaatttatcatgcttaccacaagccagcgtgggcaacggctgggctcttgtagtgtagcttttctctagccgacgcatccaggcaagggtgggcgtgatggagtttggatgggccctgcgacagcctatgcggcttccggattcacctaggcacgagaggggactgcccgtcgcccgttggggacgggggtgaaacctgaggtgtggtgtgcttggctagagggggttatgcgaagggtcctgtcacggcctctttccggtatgtcgtggtggcatgtcggcgcacggaaacgtgtcgtggggctgtgtcttgtgggtacagttgtacacctctgatcagagtaaaactattcgaatagccgtgcccgcggttatgggcgatcaaccagattcaccgtgattagactcaccctagtttagtttaatggaattggatagtacaggtggatggttgggcctgttacaacgtggtgtaacgttggacagtggatgattaatattgcttaattattacaactgttttcgctttcaactcctgtttataaatgccagaatttatgcaaatgaacctgtttagctatcccttgttaaatccctgcatcataccccccattcctgtatgacttgctgagtacagtggatagtactcagtcttgctctattttccccaaccccagagtacgagtatgtgtccgatggcgggttctccgaggagtaggcttcgtccgtgccgtcgagggtgcctgtggagtggtgttcccgctacagttcaagtccaggcttagccccagttatcttttgtttttccgctgcatttatgtaagacttttataatgtttgtaagacgtgctgtatgtcaactttgtcgtgtgtgtaccccggtcggttctggacgggagttttaatgcacattctgcttggaattctattcaggaatttctgggcgtgacagtacgctatccaggacgttggtgtttctctctcctcacgccaaaacgcaactacgtcccctcaacctagctcccaacaacatccggaaccaccaagctctctcgtcgtcaaccattgccattgtcgccagccgccgaagaccggtgtacgtagccatggagaagtgaagtggagaggaaggggtgtaccggatttaggggtgaagctcagcccctcgcgtcaagccacccgagcagcatcgtcccctccatcgctcctccacacgcgcgtcccctccatcgctcctccacatgcaccaagctttgccgtgccgccacatcgcccctccgttgctcctccacacccaccgccggctgccaccgccgcctccgtgcccgcgccagccggccgtcgcctctacctccgcgcccgctgcttgcctccttcgtgccatgccgcttccgctccgcccgcctggccgcatcgctccgcccgtgctggggaacgatgtttatccgttctttttttttctttgctttcacagtaaatgtggtagactcttttttattaatcaaccacaatatacgtgagagattaatttgtaactataatgtgaagcatgtttagcgaataacattattatttttaactaatccaggcttaaaaaacaaatacatagcaaagttggttaaaagatattattaaaatatatgaaaggtgatatggaggatgtaatatggatgatctgttggagtaggaataaatatggagaaagaatctttttgatggccatccaaactgagatatggaggctctgtttaaatcccaccctaaaatttttcacccaatcacatcgaacgtttgtacacatgtatagagtattaaatgtggaaaaaaaaccaattacacattgcgtgtaaattacaagacaaatcttttaatcctaattgcaccatgatatgacaatgtggtgctacagtaaacatttgctaatgatggattaattaggcttaataaattcgtctcgtagtttacagacggattatgtaatttgctttgttattagactatgtttaatacttcaaatatgtgtcgatatatctgatgtgatacgctaaattttacgtatatgatctaaacacccctggagttccgaatttatatgagctgctaggcaggctctaaacactgcctacatcctcggcatgttgccagctgactagaaaaaaaaaacagagagagggggcagaactccgggtcccagagaccaactgccagttcttgatagtactgcaccccagttttaaccgggaggtaactccttgcacgaacagtaaaagctttactacagtaagaaacagtagaattttgtgctttcagttcacatgcggcgttgcaagtccagctaaacaacaacgatgatcaattctcatccacactactgcgagtctgcgaccaacacggtgcccaaaaaatttgctcctgccacatgtgccaatccatcttggatttgatccccaaccagctggccagcgagccaacagtgcccacactactgtagcgttggtgcaccattaggggaacacaaacttaaacctatgttttaaacagccggttaaaacatttgatgatcggtcccccacctttcatccataaattagcacctaagaatccaaacatgtgagctaatttagggctaaagtgaattagccccccaacaaaaattagcccttctaagagatgctaatgggactaattttttgtaggactaattagcccatggatctaaacatgttaccttaggataatatttagtctaccaattcaagacaaacattagctcttaaaattaaccctagactaatcttccaaacagggcccaggttaaatgaggttaacgaggctaattttggttaaattatacataaatacgagtagctagatcctcttcaatcaactattttcaaagatagcggtagctatatccaaagatttaaaatcttatttattatgcacagttttctccgtgtttctctctagagtaaacttatttaaaaaaatattgcatacgagggtttagggcctctttgaatcacagggttgaaaaatatggaggaaaaggaaaaacaaaggattttgacaagaattgaagcgtaaaacagagggttgcaaaacacacgaaaaacacattaatggccgtttgattggagagcacgacaaacgcaggaatcggatgagagagatagactcaaaggaaattaggaaattttccaaaaggttcgagttcttgctaaattttcttcaaaatctacatgcaatatgtcattccacaggaatttcataggatttggagaacttcaatcctttgaatcgaagggccaaatagaaaaatttctataggatttgaatcctataaaatttctacataaatcatttgattcaaattcaaagtggcccttacagttgtggatttatatcatgactaaagttaggaatctacaatagaaatttactaaaattcttaatgataggatatgtgggggtatggaatatattacctcctccatttcaaaatatatgtatttttacattctttagctacaactaaggtagagtaaaaaaaatattttagtcacttgtgatcaaattttaaattgcttagattatatataggagtattctcgaaatatctcatcagctgaaaatgtataaatctccttacgttggaatcaaatgcctggaaaatacttatatcatgatacagattttgaatctaaaagattctaatattttgtaacaaggggttatgttatactccctccatcccaaaacataacaatttttagcccttagaatttgttccaaaatataacaacttctccaccaacattcttttcccaaccagttacaaccctccaccattcacttttcttacatacctctactactaattcaatcataactttccaccactcacttctacctacttaatcataattcttcaccactcacttctatttctatctattttcttaatgattatgtacaactctaaaattttttatactttgtaacggacatagtaaaaatttcttcagagagcccccaaagggggtagatagggtggttggggccccaagaggaaacacacactgtgggcactgccacacggcagccgtgctgttcactcatggtggcctgttgcgttgcgtctcctgcgctgcgtcccccccatctctctctctctctctcgctgcaccgcgatgtacaggcgtacagctcccaacccagcccgtctcctctcctctctcgctgcacatttcagcccctttttcacactagctataagctgagtagccacctagccatagcttctgcgtgatttgataactggaggcaggtcctgttgcccttgctgttgcagctgtggctcgctgcctgcactgcagtgacgctgacgccctctccctccccgcaagcactgtgctccaaagatgactggccttgccgctgctcctccatcggtagcagcacactattcctactagcatctctagtagcctatccacataccaggattttttaaaccgtcgggatcagggttactacgctccggcggtaagcacggttcccacgtggtaaccgcggtaattgtaaaaaaccatgcaaaatttatcaaaaatttaaattatttttaaaatttatttaaatttaaggaggttaccgcggtatttatattaccgtacccccgcggtaagcccggtaaccacgcggttaccggcggtaagggaaaccctgccacatactactctaccctgccacatactactctacgccaaaattttagtaacaattatagcaaagaaaaatctagtagactctccaactggatggccatgccatctagctggccaaatccctcccctcgatggtcaaacttggccagccaaaaatcacttgtcaacggtgtgcctcacgtgctattcctcccctcttctctctgttgtgctgatggataagaggcgtcgacggtgaccgttgggttaagctaggacggtgcagtgctggacgtgaggaggcggttgtgccggagcctaccccttggcactggcggtgttgggagggggggttgggcggtgggagatggcaaagcgacactgggttgcgtgagaggcaaacggtgctaggttgtcggagtccacgaattagagagtgggccaattcgtacagcggcgcgcgcggagggtggccttctcgaggtgaagtcttgactcctatgctgaggtcttggatcagccataaagctctggtcgacgtcatcgccaaaacccaccccttcccctccatcctctccgccgccacgccccctagataactattcacccttcgttcggagcccaccactgtctctactctctaccatcatagcgcctgagaatagttatgagagggcaaggacggtagggtgggtgggaggagaaagacgaaggagggagaaagaaaaagtaaagagagactgacacacgggacccactatccgattggattgtacagtgaagatgttgttggagtgaaagtcaaatttgacttttttttaagcttgtcaaatggatattttgagatgctgtcggatatactctaagctaccacatggtgtggccatacatgcgcgagtaccccatggttaacacagtaagctcatacggtaaccattttagttattttttaccgtattaacctcgtgccgtaaccatcttagttttcaaatcataccattttttctcgctgaccacggtagccatgatatcattgggtagaggtaacttctaccaaagcaatagtgaatgtgtaccatggagagaagccaaagccgtgcacgccggagaaaattgatgttatgccatcgtcggaaagggatttcgctaaaatatcataacacatttaggattcaataaaatggtactctcaaataggatcaacacgccgcaatgacatttctttccttcttaatttaatgatttttcatgtttgctttggcaaagctaaccaaatgcccttcttcctcttgatccctttttttgtctcattgatgagagctcgacgaagtagtagcaccaccatggctagggaagaggtcgtaaatcaaagatatcatgcccgcgcgctcgtcgatagccgcaacaggcgtcttctactcttcaacgtggcggggaggtcctccaagatgaacctgatgcctttaccagtggtacaaccctcgcacaagagcgaacgaagatcgccggccttggtctccactacctccctcacacttcgtgcaaggacaaagagggacacgaactggaggttcttgaggcagttgtgcctgccctgtccatcgccgccactcgcaaccacaccaagatgtgaagcatccgttgtctggacctccatggccacgccaacgcaaacacccttgtccaatacttttcaatagtgtcgccacatggtcgaagctccatcaccgcctccgatctaagcctcaccacccttatcccaatgttcatctcaacatcctttcctcttctacttcccctacaagatcgagtaaaagtctcagcaaagattcacaatataatttctcttttatttctcctacgagatcgagtagaggtctacgtaaagatttaataagtgcccattatttaatagtaacattttattgaatctcgtttagagcaggtacaatagcaggctacaagtcagctataagcacatgtggaagagaaaaaagaggagagagaagcaaagcaggctacaaatttgtagccagctgcagcacagactccaaaacgttatatgtgtacgagaggcgggaccatatattaatggtgtagtatatgtttataagtaactattgtatgaataaactattagattggctagctataatagtaagctccaaatcatctatagccaatataatagccaattcatacaatagttgcttactatactattaatacctggtcccaccaatcacacacacgttaggtcttggagtctgtgctgcagctggctacagatttgtaacccgctgctcttctctcttcctttatttttttaaagagtaaatttcataaaactacagatactttgcatgatctatcacaaaactacagattttagaactagtttcacaaaactacagatttaatgtgttcatttatcacaaaactacatgtactttgaacactatatcacaaaactacagatttaagaacttgtttcacaaaactacaggtttagtatcttcatttttcacaaaactacaaatttaatatcttcatttatcacaaaactacagattcagtgtctccattctcataaaactacacgttttaataatgctaaaactattattaaaacgtatagttttatgagaatggagacactgaatctgtagttttgtgataaatgaagacactaaatgtgtagcattgttataaacgaatacactaaatctatatttttgtgaaacaagttcttaaatatgtagttttgtcatagattgtacaaagtacctgtagttttgtgataaaacgaagacactaaatctgtagttttgtgataaattgtacaaaaatacctgtagttttgtgaaatttactcttttttaaaatatgtttatagctggcttaggccatttaaagtgcaaggacgtggcgtgtggcaatgtagagccacgtaggcaagtcgcttgcgtggaggagagaggggagtggggaccgttcccaacccagcttcgtgtgaccaagtttggccacacgggccaaacgaacctcagcaacttttgtcagaaagaaaagaacctccgcgagaaacaagaaagcgagagagggagagaaaggaggctcgtcggagtaggggcgctcggggtatggggctcggcggaggctcgctggagtaggggccgccactgcgtggggctcgccggagtaggggcgctcggggagcctcccgagatccgccgctcagcggcgccgccgtcttccgggcagagctctcgaagctcgctctcctcgcgcgccggtggcgttggcggggcccgcgtgtggctacgcagctccggtgctgcgcctccaccgtcgacgacagcgccgcttggcgctgccgccgtcttccgccgcgccgctggacgccgccagatctgctgctcgtcgccgcgtgggccgctccacccggttggaggaggagaggcggcgccgcgcttgggctgccccaccgccgagctctgccgcgccgttcgccggtgctgccgagctccgccgcgcctgccggagcacgctgccatggccgccctggagaagacacgagagaattaggtggagggtgggggaagggtgagattttttatattatctatgggttccattataaattttctaaaccacacttatactgtgggtgcagtgtcatttagagttcccaaaccacctatgttgcagctgtggtataacaatttgctaggacgcattgctactgcccttgtaccctgctataagaagataaccaatgacatctccactcgattttctcggcgcgcgtgtgagggtgtgaggataatttttattttaagtggtttttaagggcggagagagagagagagagagggcaccgcactacttctacttgtgtgtgtgtcgctcgctgggcttcgccacctttccgtctctttcctctctcttctctctccccctctcctggaggagagagaggagaagaggagggggggccgcgccaagagccacgcgcgctacagtctccttcccacccgcgaccgcgagcaatggagatggccagtggaggaggcgccgccgccgccgccggcggcggagtaggcggcagcggcggcggtggtggtggaggggacgagcaccgccagctgcacggtctcaagttcggcaagaagatctacttcgaggacgccgccgcggcagcaggcggcggcggcactggcagtggcagtggcagcgcgagcgccgcgccgccgtcctcgtcttccaaggcggcgggtggtggacgcggcggagggggcaagaacaaggggaagggcgtggccgcggcggcgccaccgccgccgccgccgccgccgcggtgccaggtggaggggtgcggcgcggatctgagcgggatcaagaactactactgccgccacaaggtgtgcttcatgcattccaaggctccccgcgtcgtcgtcgccggcctcgagcagcgcttctgccagcagtgcagcaggtcactctctcactcacctcgccattgctgatgtcaccactgcttttgctttgctttgcttgctctccctcctctttcacctatctctcttgtttatttgcttcttgttcttgtttagtgctagtacatgtgttgttattgttgtgccgttttgtcttttgggttattgtgttgttgttactactcgttttactataggtttttaaggtttatgagcacggccaccacattagatgcactgtcaagtggtgtgtgtgggacctttcctgctaaaacaagctgatttcaactctctgaaacttcctgcatttcatctatttttatctttgattgtgttgggagtactacactagtagtgttaatattttgactggtgcttatgagatttttaagttggtaggttgatgaggaaaatactcctttatatggttgagtgatgtgacttgcctgtctgcctgcctgcctgccgctttgcataagattcctctgtgttagtaagagccactgtttatttgtactggtgcttactctacttagttaattagccattagctataaaattccgttgatgttgcaagcttagcaatggccacggtaagaatgggagagagaagttggctaaagctgttgctttgtagtttgtactatatatgtgtctttgtgttgcaagatatgcaactcctactatgctgtgacttgagctcaaggttttcagttatctatagatccttactactactgagcatactaccacttctgtatggtagcatatggtagcatagtccaagttccaacgcctcgccagttgttcataatctatactaccacttctgtgcatttgttacttttatttaatagtttgtctcattagctgacaagcatatgcctgttttgatatctgcccctcttgtaatagtctatggatagcttggactgtttgatgctttaattttttactagcaacacttagggcccctttgaaatggaggattagcaaaggaattttggaggattcattttcctaaggattttttcctatagagccctttgattcatagaaagaggataggaaaacttccgtaggattgcattcctatgatcaattccataggaaaataagcaagaggttagacctcttgtgaaactttcctttgttgagtgtatcttgtggtataatcaaagggctcttctctccatttcatgtgttttcaattcctgtaggattggaaaaacatacaacttcaattcctacgtttttcctattcctatgtttttcctatcctgcgtttcaaaggggcccttaaggatgaagggaagtaagagaaacatactagagaatatgtagtagtatttctacattccatatttgtagcactagcccacaaatatctttgccttgtacttacttcataccagttccccccttttcagagcaaaccaacaatttctgttgccttatatatctagtgtcttcgtactaatatatctgttccaaaatgtacctgtccaaattcatagctagaaatagctttatttaggacggaagtaataactgttgttagagacttggttcagacttttggttatgttgaggctactatcatttcctttacgggccaaattactacaaatgagaattcataaaaatgtcaagattttatgattgttgtagctttatttaggacggaggtagtaattgttgttagagacttggttcagacttttggttacgttgaagctactatcatttcctttatggtcaaattactaacaatgagtattcataaaaatgtcaagattttataattgagctgtgccagtgctaagtgtgtcactatctgatgccataatgcatcattataaaagccagatggaccattagcttttatgtgtaggacacctgccgtccaattagatggataaccatctagtgtttgtgtactgttattttaagcccgacatctcacaactccatgaatgattacagtcttcctttcacatggtgtccttttgttgtgttaggaatagcattttttatttatgggtgtaattatgaaaggcactaggagagttgctgctttatcttgatgggatttgtagtaataccatctttaggatgacaagaaatcttgttctgagttagcatgggctgccttttgacctgagctacggtttgctatgtttggcttgcatcatgcagatctattaggataataagcatataaaagttgcttgcattgtgcattgcttgttttaccttgattcatgtaggagtaatttgctcgccatgcctcgttttgctttctgagtcaacagccaaatttagatgatgtaccttctgttgcttcaaaaactcagtcactgcacagcagcagtggataggattcagaatcaatctatccatgattctctgttcacataatatgacaggttccacctgctgcctgaatttgaccaaggaaaacgcagctgccgcagacgccttgcaggtcataatgagcgccggaggaggccgcaaacccctttggcatcacgctacggtcgactagctgcatctgttggtggtatcatcagaggctcttgttttctttgcatcttgtgtgtttgttggtaactactggttgcattcgctgatgtgttgtttgttgcgattcttgatccagaagagcatcgcaggttcagaagctttacgttggatttctcctacccaagggttccaagcagcgtaaggaatgcatggccagcaattcaaccaggcgatcggatctccggtggtatccagtggcacaggaacgtagctcctcatggtcactctagtgcagtggcgggatatggtgccaacacatacagcggccaaggtagctcttcttcagggccaccggtgttcgctggcccaaatctccctccaggtggatgtctcgcaggggtcggtgccgccaccgactcgagctgtgctctctctcttctgtcaacccagccatgggatactactacccacagtgccgctgccagccacaaccaggctgcagccatgtccactaccaccagctttgatggcaatcctgtggcaccctccgccatggcgggtagctacatggcaccaagcccctggacaggttctcggggccatgagggtggtggtcggagcgtggcgcaccagctaccacatgaagtctcacttgatgaggtgcaccctggtcctagccatcatgcccacttctccggtgagcttgagcttgctctgcaggggaacggtccagccccagcaccacgcatcgatcctgggtccggcagcaccttcgaccaaaccagcaacacgatggattggtctctgtagaggctgttccagctgccatcgatctgtcgtcccgcaaggcgagtcatggaactgaagaacctcatgctgcctgcccttattttgtgttcaaattttcctttccagtatggaaaggaaattctaaggtgactggcgattaatctccctgtgatgaataataatgcgcgcccttgaactcaattaattgctgtgccgcatccatctatgtaactctccatgaatttttaagtatcagtgttaatgctgtattgtcgaggacttctgctcgatatgttatttctcttatgttgttcatcatgaatctttttctgcttattattctggtgccgggttgtccttaccacagaagattcagtttcggttggcgagagtaaacaccttccctggttgtgacaaaagctccaaccttttcacttctcggcctgtatttgatcttccccttctgacgctgttatactacttttaagcctgtatgtttccagccttccaggtgaagggccatactgaagagaaaacatgctttcagggtttgatgcattgtgtactttacaagtgtacttaagattttgtacaatttatatatgtacctgctctgctgctgagtattgtaggaaagaatcagttcgaagggcgtgtgttcatgtaaagtgagaccacatgcacagcgtggatttgcagcatgctctctgcaccagtggtgttctgttgatgcctttgatgggctggctgaggtgagaggaggatgatccatgttggcagcttcttcactctgaaaaataaaagagaagaaatgttcagatttgcagacaagtggagagcagtgatatattctacaataaaacattaccaccttgcttttctgtgatgatagatactccatggaattttgcatcaagcatctcttgttttccagccactgtttgctgggttgttgcttcaatttcgtcccaattgattggtcacctttggttgtgacttgagagcactgagcactgaaacttttgctgtcagcaggcaatgcacctcatccatgtcacgacagagggagagagcccacataaatggccaaagaggacacatacagtggcactgatgcagtcattgcaacataattgacatcatgctaaacagtggtgtaaccatatgttagctagcctgtgatcagcaaacagtgattatggatcttaatgtcacatgcaagatttgacacagttgtaaaaccatcattgcattgaagatagatcccagcaacaggtgtatgatgtattgctagaatgaatcaaaaatatcagtgccatcctaaacacagtactaccaacttgaacagttatcaccgtgattggaaaacagaaatgtataattgctttggcgccatctgcttatcattatcatatgtcgcagatcacttgttccattgacacgactctttttcactgtgaggagaggcaccttgatttggacttttcaagagctgtagcaagggctcctttgaagccttctacatggaggagcagagcataccatatgcagaactgtaaactcttcctgaagctttccagttccacccttgtagctttaagctgccgcaagagaattatcatttctaacattgagatgtgatactgaaatgtgaaaggtgattcgcagtataggtcccaaaatatcgtttacagcaacttgcaaatcctgcatgatacagttaattcatcaaaatattagaccattagtactacagtctacaaataccccttaactgaacatgtatgataaggacaagattctgaagctccagtgcatcaggaatccaacgcagtatgcaaatcattactgaacaagattcctgcacttacagaatcatcacctgttgtaacaaggaccattctttgttgccccagacacagcgaattaatggtcatcttcatttggcccaggacattcatttgccaatgcttctgctgattcatactgaaaaggggacaatgcgtccaattttaaaagcatggaagatgctataaaagatcaccctatttaaaatgcagagaaaaccaaagatccaacatgatatggtaatcacagattcccaacagtaatgccgtccagtaggcagtaggggcatgcacataaacactagtactatgtagagctgaagcttatttccagaatgaagctgaccttgcaaccgcaataaaggcaatagtagtgttcatcgcgcagcttagccaatatattttgcaaatcctgcaagaataaacacaggtcaatctcgtcctttcagcaaaatttgcagtcttgcataagatttctcagataaaaaaggaagtctagacaagaatatgaaggaaagaatgtgcaaacataagtatattcataattcaaagttcgagctatttccattgtcacaacataatatgtgtaaccaatagctggtaagcatttacggttagcccttcataggaaccaaataggcacggaggcaattgcgttactaataccagttatcatgctcgtcacaatggaaaattgtgatgaaatgcctaaggtcctatgcaatatatcctttatttctatatagttacaacccactagcagcatttatttctaaaaagctgctccacatttactcatgtcagcagcacatttgaaccattggatcaatcgttccgtgttgataaattgttggatc

(19) United States
(12) Patent Application Publication
(10) Pub. No.: US 2012/0185959 A1
(43) Pub. Date: Jul. 19, 2012

(54) GENE REGULATING NUMBER OF PRIMARY PANICLE BRANCHES AND USE THEREOF

(75) Inventors: Motoyuki Ashikari, Nagoya-shi (JP); Hidemi Kitano, Nagoya-shi (JP); Kotaro Miura, Nagoya-shi (JP)

(73) Assignee: NATIONAL UNIVERSITY CORPORATION NAGOYA UNIVERSITY, Nagoya-shi, Aichi (JP)

(21) Appl. No.: 13/384,449

(22) PCT Filed: Jul. 16, 2010
(86) PCT No.: PCT/JP2010/062119

§ 371 Date: Mar. 29, 2012

(30) Foreign Application Priority Data
Jul. 16, 2009 (JP) 2009-167734

(51) Int. Cl. A01H 1/02; A01H 5/00; C07H 21/04; C12N 15/82; C12N 5/10; C12N 15/63

(52) U.S. Cl. 800/260; 435/419; 435/320.1; 536/23.6; 800/278; 800/298

(57) ABSTRACT

Isolation and identification of a gene associated with the number of primary panicle branches in a plant, and use of the gene. A gene encoding a protein having the amino acid sequence set forth in SEQ ID NO:2 is isolated as a gene associated with the number of primary panicle branches in a plant. By using the gene, it is possible to readily create a plant which has a large number of primary panicle branches and a large number of grains.

### TECHNICAL FIELD

The present invention relates to the isolation and identification of a gene regulating the number of primary panicle branches associated with yield increases in the seeds and foliage of a plant, and to a method for improving the yield of a plant using such a gene.

### BACKGROUND

As the human population grows, we are seeing global food crises triggered by environmental contamination, global warming and other factors. This situation has intensified the need to increase the yield of those grains which serve as food staples. Hence, the breeding and diffusion of high-yielding rice is today an important issue.

The genetic characteristics of cultivated varieties of rice are determined by the sum total of the mutated loci in each variety. The loci which exert an additional influence on a specific trait in this way are called quantitative trait loci (QTL). Accordingly, the genetic characteristic of a high number of formed grains in a particular variety may also be regarded as being determined by the quantitative trait loci. Genes associated with changes in the number of ripened grains have already been isolated and identified in rice (Patent Literature 1).

### CITATION LIST

### Patent Literature

Patent Literature 1: Japanese Patent Application Laid-open No. 2007-49994

### SUMMARY

Various other varieties of high-yield rice also exist. In each variety, the QTL to be identified differs according to the type of trait contributing to a high-yielding ability. By combining different traits which contribute to a high-yielding ability, further increases in yield can be expected.

It is therefore an object of this invention to isolate and identify a gene which regulates the number of primary panicle branches in a plant, and to make use of such a gene.

In order to isolate and identify a gene which regulates the number of primary panicle branches in rice, the inventors carried out QTL analysis combined with positional cloning. As a result of an enormous amount of experimentation and analysis, the inventors were able for the first time to isolate and identify a gene which regulates the number of primary panicle branches. That is, the inventors have found that when the expression of this gene is suppressed, the number of primary panicle branches tends to decrease, and that when expression of this gene is enhanced, the number of primary panicle branches tends to increase. In addition, when the identified gene was inserted into other cultivars and the expression of a protein encoded by the gene there induced, a transformed plant that had acquired the ability to increase the number of primary panicle branches was obtained. Based on these findings, the following teachings are disclosed in the present specification.

Accordingly, the present teaching of the specification provides a transformed plant in which expression of a gene encoding any one of proteins (a) to (f) below is enhanced:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity; and
(f) a protein which is encoded by DNA having at least 70% identity with the base sequence set forth in SEQ ID NO:1, and which has a primary panicle branch number-increasing activity.

In the transformed plant disclosed herein, the protein is preferably from a gramineous plant. Moreover, the transformed plant is preferably a gramineous plant.

The present teaching of the specification also provides a vector which, in order to enhance expression of a gene encoding any one of proteins (a) to (f) above, carries at least a portion of the gene. A plant cell to which the vector has been transferred is also provided. In addition, a transformed plant containing the plant cell is provided as well. Also provided is a transformed plant which is a progeny or clone of the foregoing transformed plant. In addition, a propagation material for the foregoing transformed plant is also provided.

The present teaching of the specification additionally provides a method of producing a transformed plant, with this method including a step of transferring in use of the foregoing vector the gene into a plant cell and regenerating a plant from the plant cell.

The present teaching of the specification also provides a method of producing a useful crop, with this method including a steps of cultivating the foregoing transformed plant, and harvesting the transformed plant or a portion thereof.

In addition, the present teaching of the specification provides a method of regulating a yield of a plant or a portion thereof by regulating a level of expression of a gene encoding any one of proteins (a) to (f) above in the plant. The teaching also provides a chemical agent for modifying a yield of a plant or a portion thereof, the chemical agent containing, as an active ingredient, a gene encoding one of proteins (a) to (f) above.

The present teaching of the specification further provides a plant which carries a DNA region, the DNA region including a first DNA encoding any one of proteins (a) to (f) above and, upstream of the first DNA, a second DNA (g) or (h) below, at a locus where the first DNA is originally positioned or at a position corresponding to this locus:
(g) DNA which has a base sequence set forth in SEQ ID NO:3;
(h) DNA which has a base sequence having, in the base sequence set forth in SEQ ID NO:3, one or more substituted, deleted, added and/or inserted base, and which has an ability to enhance expression of a protein having a primary panicle branch number-increasing activity.

This plant is preferably a monocotyledon, and more preferably a gramineous plant. This plant may be a plant obtained by crossing. This plant may be, at the foregoing locus, homozygous for the foregoing DNA region.

The present teaching of the specification additionally provides a method of producing a plant, with this method including a step of crossing a parental variety of plant which carries a DNA region, the DNA region including a first DNA encoding any one of proteins (a) to (f) above and, upstream of the first DNA, a second DNA (g) or (h) above, at a locus where the first DNA is originally positioned or at a position corresponding to this locus with other plant so as to produce a new variety of plant which carries the DNA region at this locus where the first DNA is originally positioned or at a position corresponding to this locus.

In the foregoing plant production method, the new variety of plant may be screened for using, as a marker, DNA containing at least a portion of the second DNA.

The present teaching of the specification also provides a breeding marker which contains at least a portion of the second DNA.

The present teaching of the specification additionally provides a DNA fragment for breeding, with this fragment containing a DNA region that includes the first DNA and, upstream of the first DNA, the second DNA.

The present teaching further provides a DNA fragment for breeding, with this fragment containing the second DNA.

### BRIEF DESCRIPTION OF DRAWINGS

FIG. 1 is a diagram comparing panicles of NP-12 rice and Nipponbare rice.

FIG. 2 is a diagram showing the results of a QM analysis cm the number of primary panicle branches in NP-12 rice and Nipponbare rice, and QTLs were detected on the short arm of chromosome No. 1 and the long arm of chromosome No. 8.

FIG. 3 is a diagram showing the cloning results for the WFP gene, which is a gene that regulates the number of primary panicle branches, and this shows identification to be possible at the promoter regions of the OsSPL14 gene.

FIG. 4 is a diagram showing the results of OsSPL14 gene expression analysis.

FIG. 5 is a diagram showing the increase in the number of primary panicle branches owing to SPL14 gene transfer.

FIG. 6 is a diagram showing the genotypes in four kinds of BC₂F₂ progeny obtained from Nipponbare and NP-12.

FIG. 7 is a diagram showing the measurement results for the number of primary branches per main panicle in four kinds of BC₂F₂ progeny.

FIG, 8 is a diagram showing the number of grains per main panicle in four kinds of BC₂F₂ progeny.

FIG. 9 is a diagram showing the number of grains per plant in four kinds of BC₂F₂ progeny.

FIG. 10 is a diagram showing the results of an evaluation, based on the QTL on chromosome No. 8, of the number of primary branches per main panicle for a heterozygote and for homozygotes of Nipponbare and NP-12.

FIG. 11 is a diagram showing the results of an evaluation on the level of methylation in a 2.6 kb region for both Nipponbare and NP-12.

### DESCRIPTION OF EMBODIMENTS

The present invention relates to a gene for regulating the number of primary panicle branches, and to the use of such a gene. The invention is based on the first successful isolation and identification by the inventors, in the manner described below, of a gene which contributes to an increase in the number of primary panicle branches, thereby enabling an increase in the number of formed grains and thus an increase in yield to be achieved.

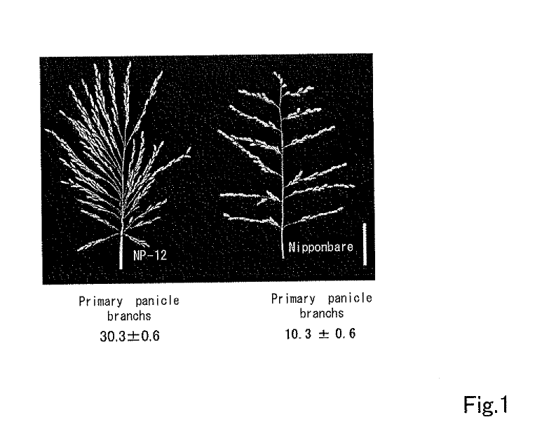

The inventors focused their attention on the high-yielding Indica-type rice strain NP-12. The rice strain known as Nipponbare has about 150 grains per panicle. By contrast, NP-12, a stock strain of rice preserved at Nagoya University (the seeds of this plant can be acquired from the Nagoya University Bioscience and Biotechnology Center), has about 240 grains, and also has about three times as many primary panicle branches (the number of branches that emerge from the rachis) as Nipponbare (see FIG. 1). The inventors surmised that the large number of formed grains and the high yield of this stock strain of rice are due to the high number of primary panicle branches, and successfully set out to isolate a gene that regulates the number of primary panicle branches.

The present invention, by modifying a plant in such a way as to enhance expression of the gene that has been newly isolated and identified by the inventors, makes it possible to obtain a transformed plant having a high number of primary panicle branches and an increased yield of seeds and foliage. The gene discovered by the inventors was a gene endogenous to gramineous plants; although this gene is expressed in NP-12, a similar endogenous gene was carried in Nipponbare but was not being expressed. It was found that inactivating this endogenous gene or inhibiting its expression lowers the number of primary panicle branches, and that, conversely, enhancing the expression of this endogenous gene contributes to an increase in the number of primary panicle branches.

The present gene is useful particularly in the fields of agriculture, energy involving the use of biomass as a raw material, and industrial chemistry. For example, the increased number of formed grains of seeds resulting from the increased number of primary panicle branches allows the increased yield of crops such as grains.

When using the gene isolated and identified by the inventors to create a plant, it is preferable to do so by transformation. The period of time required for transformation is very short compared with gene transfer by crossing, enabling a primary panicle branch number-increasing ability to be imparted or enhanced without accompanying changes in other characteristics. Moreover, because genome synteny (gene homology) is very well conserved in grains, the gene isolated by the inventors is likely to be used in the breeding of grains such as wheat, barley and corn.

The gene discovered by the inventors is thought to be utilized in plants other than rice, including other gramineous plants such as wheat, barley, corn, sugar cane and sorghum, and is likely to be of wide use in the fields of agriculture, energy and chemical industry.

This specification also discloses the use, in producing a plant having an increased number of primary panicle branches and an increased yield, of a DNA region of the gene isolated and identified by the inventors within chromosome No. 8 of the stock strain of rice NP-12 preserved at Nagoya University which includes also a region upstream of the identified gene.

Of this DNA region, the region upstream of the identified gene, at an original position on the chromosome, contributes to enhancement of the expression of the identified gene, operatively linked downstream thereto, which plays a role in increasing the number of primary panicle branches. Therefore, by transferring this DNA region (which, inclusive of the upstream region of the identified gene, may be referred to as an allele) or a region upstream of the identified gene to a chromosomal position where the identified gene is originally positioned or upstream therefrom, expression of the identified gene is enhanced, enabling an increase in the number of primary panicle branches or the yield to be attained. Such transfer of specific DNA onto a chromosome is achieved more easily by crossing than by gene recombination using genetic engineering techniques.

In connection with the teachings presented herein, the gene regulating the number of primary panicle branches, the expression vector, the transformed plant, and the method of producing a useful crop are each in turn described below.

Gene Regulating the Number of Primary Panicle Branches

The gene which regulates the number of primary panicle branches (sometimes referred to below simply as “'then”) encodes a protein which induces the number of primary panicle branches (sometimes referred to below simply as “the protein”). The OsSPL14 gene (NM_{—}001068739 REGION: 124 . . . 137) of rice (Oriza sativa) was first identified by the inventors as a gene associated with regulation of the number of primary panicle branches, both by QTL analysis on the number of primary panicle branches using NP-12, a variety native to Indonesia and a stock strain preserved at Nagoya University, and by positional cloning. Up until now, nothing has been reported on the function of the protein encoded by the OsSPL14 gene. So long as this gene encodes a protein having a primary panicle branch number-increasing activity as described above, the gene may be either one prepared from a native origin or one prepared artificially. Illustrative examples include orthologs and homologs of the OsSPL14 gene, and versions of the OsSPL14 gene in which mutations have been artificially introduced. The gene may be, for example, genomic DNA or cDNA. The gene and the protein encoded by this gene may be primarily from the plant world, and in particular from a gramineous plant. This gene is one that was isolated from rice, but is thought to be present in plants which similarly have a large number of primary panicle branches. The gene is preferably from a monocotyledon, and more preferably from a gramineous plant. Persons skilled in the art may suitably acquire information relating to such genes and proteins by accessing the home page of, for example the National Center for Biotechnology Information (NCBI; http://www.ncbi.nlm.nih.gov). The protein is described below.

The Protein

One form of the protein is a protein having the amino acid sequence set forth in SEQ ID NO:2. Another form of the protein is a protein having a fixed relationship with known sequence information such as SEQ ID NOS:1 and 2, provided such a protein has a primary panicle branch number-increasing activity.

Yet another form of the protein is a protein which has an amino acid sequence having, in the amino acid sequence set forth in SEQ ID NO:2, one or more substituted, deleted, added and/or inserted amino acid, and which has a primary panicle branch number-increasing activity. The phrase “primary particle branch number-increasing activity” refers to the characteristic of increasing the number of primary panicle branches, compared with before, by expressing this protein or by promoting such expression. The degree of such increase is not in question, so long as the number of primary panicle branches is increased. Specifically, when a transformed plant having an enhanced expression of DNA coding for this protein is cultivated and the number of primary panicle branches is found to have increased, the protein and DNA may be said to have a primary panicle branch number-increasing activity. When the number of primary panicle branches is unchanged or substantially the same, the protein and DNA cannot be said to have a primary panicle branch number-increasing activity. Determination of the number of primary panicle branches may be carried out by the method subsequently described in the examples.

Amino acid changes with respect to the amino acid sequence set forth in SEQ ID NO:2 may be of any one type, or may be a combination of two or more types, from among deletions, substitutions, addition and insertions. The total number of such changes, although not particularly limited, is preferably at least one but not more than about ten, and more preferably at least one but not more than five,

Preferred examples of amino acid substitutions are conservative substitutions, such as substitutions within the following groups: (glycine, alanine), (valine, isoleucine, leucine), (aspartic acid, glutamic acid), (asparagine, glutamine), (serine, threonine), (lysine, arginine), (phenylalanine, tyrosine).

Another form of the protein is a protein which has an amino acid sequence with at least 60% identity with the amino acid sequence set forth in SEQ ID NO:2, and has a primary panicle branch number-increasing activity. The identity is preferably at least 70%, more preferably at least 80%, even more preferably at least 85%, still more preferably at least 90%, still yet more preferably at least 95%, and even more preferably at least 98%.

In this specification, “identity” or “similarity,” as is commonly known in the technical field to which the invention relates, refers to the relationship between two or more proteins or two or more polynucleotides as determined by comparing the sequences thereof. In the art to which the invention relates, “identity” refers to the degree of sequence invariance between protein or polynucleotide sequences, as determined by the alignment between protein or polynucleotide sequences or, in some cases, by the alignment between a series of such sequences. “Similarity” refers to the degree of correlation between protein or polynucleotide sequences, as determined by the alignment between protein or polynucleotide sequences or, in some cases, by the alignment between a series of partial sequences. More specifically, these are determined by the identity and conservation (substitutions which maintain specific amino acids within a sequence or the physicochemical properties of the sequence) of the sequence. The similarity is indicated under the heading “Similarity” in the subsequently described BLAST sequence homology search results. The method for determining identity and similarity is preferably a method designed to give the longest alignment between the sequences being compared. Methods for determining identity and similarity are furnished as publicly available programs. For example, determinations can be made using the BLAST (Basic Local Alignment Search Tool) program provided by Altschul et al. (e.g., Altschul, S. F.; Gish, W.; Miller, W,; Myers, E. W.; Lipman, D. J.: J Mol. Biol., 215:403-410 (1990); Altschul, S. F., Madden, T. L.; Schaffer, A. A.; Zhang, 1; Miller, W.; Lipman, D. J.: Nucleic Acids Res. 25:3389-3402 (1997)). The conditions when using software such as BLAST are not subject to any particular limitation, although using the default values is preferred.

With regard to the base sequence encoding the amino acid sequence set forth in SEQ ID NO:2 or an amino acid sequence having a fixed correlation as noted above with this amino acid sequence, owing to degeneracy of the genetic code, at least one base of a base sequence encoding a given amino acid sequence may be substituted with another type of base without altering the amino acid sequence of the protein. Accordingly, the gene also encompasses genes with base sequences that have been altered by substitution based on degeneracy of the genetic code.

The protein may also be a protein encoded by DNA containing the base sequence set forth in SEQ ID NO:1. Yet another form of the protein is a protein encoded by DNA which hybridizes under stringent conditions with DNA having a base sequence complementary to DNA having the base sequence set forth in SEQ ID NO:1, and which has a primary panicle branch number-increasing activity.

“Stringent conditions” refers herein to conditions under which so-called specific hybrids form and non-specific hybrids do not form. Such conditions are exemplified by conditions where a nucleic acid having a high base sequence homology, i.e., a complementary chain of DNA composed of a base sequence having at least 60%, preferably at least 70%, more preferably at least 80%, even more preferably at least 85%, still more preferably at least 90%, yet more preferably at least 95%, and most preferably at least 98%, identity with the base sequence set forth in SEQ ID NO:1, hybridizes, and a complementary chain of nucleic acid having a lower homology does not hybridize. More specifically, “stringent conditions” refers to the following conditions: a sodium salt concentration of from 15 to 750 mM, preferably from 50 to 750 mM, and more preferably from 300 to 750 mM; a temperature of from 25 to 70° C., preferably from 50 to 70° C., and more preferably from 55 to 65° C.; and a formamide concentration of from 0 to 50%, preferably from 20 to 50%, and even more preferably from 35 to 45%. In addition, under stringent conditions, the filter washing conditions following hybridization generally are as follows: a sodium salt concentration of from 15 to 600 mM, preferably from 50 to 600 mM, and more preferably from 300 to 600 mM; and a temperature of from 50 to 70° C., preferably from 55 to 70° C., and even more preferably from 60 to 65° C. From the above, yet another form of this protein is a protein, which is encoded by DNA having a base sequence with at least 70%, preferably at least 80%, more preferably at least 85%, even more preferably at least 90%, still more preferably at least 95%, and most preferably at least 98%, identity with the base sequence set forth in SEQ ID NO:1, and which has a primary panicle branch number-increasing activity.

The gene encoding the various above forms of the protein may be obtained as a nucleic acid fragment by using a primer designed based on the sequence of SEQ ID NO:1 or the like to carry out PCR amplification with, as the template, DNA extracted from a gramineous plant or the like, or nucleic acid from various cDNA libraries or genomic DNA libraries. Or the gene may be obtained as a nucleic acid fragment by carrying out hybridization using nucleic acid from the above libraries or the like as the template, and using as the probe a DNA fragment which is a portion of the gene. Alternatively, the gene may be synthesized as a nucleic acid fragment by various methods for synthesizing nucleic acid sequences that are known in this technical field, such as chemical synthesis.

The gene encoding the various above forms of the protein may be acquired by modifying DNA encoding the amino acid sequence set forth in SEQ ID NO:2 (which DNA is composed of, for example, the base sequence set forth in SEQ ID NO:1) using, for example, a conventional mutagenesis technique, a site-specific mutation technique, or a molecular evolution technique employing error-prone PCR. Such techniques are exemplified by known techniques, including the Kunkel method and the gapped duplex method, and by methods in general accordance therewith. For example, changes may be introduced into the DNA by using a mutagenesis kit that employs site-specific mutagenesis (e.g., Mutant-K and Mutant-CT, both available from TAKARA), or by using an LA PCR in vitro Mutagenesis Series kit from TAKARA.

Aside from the above, by referring to, for example, Molecular Cloning (Sambrook, J., et al., Molecular Cloning: A Laboratory Manual, 2^{nd} ed. (Cold Spring Harbor Laboratory Press, 10 Skyline Drive, Plainview, N.Y. (1989)), a person of ordinary skill in the art will be able to acquire, based on a known sequence such as SEQ ID NO:1 or 2, the gene encoding various forms of the protein.

Expression Vector

The expression vector of the invention may be a vector for enhancing expression of the gene in a plant cell. The vector of the invention may carry the gene. The vector may be intended to introduce the gene as exogenous DNA, regardless of the endogenous presence or absence of the gene on a chromosome in the host cell (plant cell) and, as a result, to enhance expression of the gene. However, such a vector does not exclude the possibility of, by homologous recombination or the like, enhancing expression of the gene endogenous to a chromosome in the plant cell.

Illustrative, non-limiting, examples of the plant cell include mouse-ear cress (Arabidopsis thaliana), rice, corn, potato and tobacco cells. Examples of gramineous plants include rice, wheat, barley, corn and sorghum. The term ‘plant cell’ encompasses not only cultured cells such as suspension cultured cells, but also protoplasts and calluses. Plant cells include shoot primordia, polyblasts and capillary roots, as well as cells within a plant, such as a leaf section.

This vector, when it is intended to introduce the gene as exogenous DNA into a plant cell and induce expression of the gene, may contain a promoter capable of transcription in the plant cell and the gene which is operatively linked under regulation by the promoter. In addition, the vector may also contain a terminator which includes poly(A). Illustrative examples of such promoters include promoters for constant or induced expression of the gene. Examples of promoters for constant expression include the 35S promoter of the cauliflower mosaic virus (Odell et al., Nature, 313:810 (1985)), the actin promoter of rice (Zhang et al., Plant Cell, 3:1155 (1991)), and the ubiquitin promoter of corn (Cornejo et al., Plant Mol. Biol., 23:567 (1993)). Promoters for induced expression of the gene include promoters which are known to induce expression as a result of external factors, such as fungal, bacterial or viral infection or invasion, low temperatures, high temperatures, drying, exposure to ultraviolet light, or the spraying of specific compounds. Examples of such promoters include the rice chitinase gene promoter (Xu et at, Plant Mal. Biol. 30:387 (1996)), the PR protein gene promoter in tobacco (Ohshima et al., Plant Cell, 2:95 (1990)), the “lip19” gene promoter in rice (Aguan et al., Mol. GenGenet., 240:1 (1993)), the “hsp80” gene and “hsp72” gene promoters in rice (Van Breusegem et al., Planta, 193:57 (1994)), the “rab16” gene promoter in A. thaliana (Nundy et al., Proc. Natl. Acad. Sci. USA, 87:1406 (1990)), the chalcone synthesis enzyme gene promoter in parsley (Schulze-Lefert et al., EMBO J, 8:651 (1989)), and the alcohol dehydrogenase gene promoter in corn (Walker et at, Proc. Natl. Acad. Set USA, 84:6624 (1987)).

Alternatively, the vector may be one that is intended to induce production of the protein as a recombinant protein in host cells, such as Escherichia coli, yeasts, animal cells or insect cells. In such a case, the vector may contain the gene under regulation by a promoter capable of operating in a suitable host cell.

The vector may be constructed by a person skilled in the art using a commercially available material such as any of various plasmids known to persons skilled in the art. By way of illustration, the vector may be constructed using, for example, the plasmids “pBI121,” “pBI1221,” or “pBI101” (all available from Clontech), and using a vector which expresses the gene within a plant cell in order to create a transformed plant.

The present teaching of the specification also provide a host cell, such as plant cell, to which such an expression vector has been transferred. Also provided is a chemical agent for modifying the yield of a plant or a portion thereof, which agent includes the gene as an active ingredient. More specifically, a chemical agent for modifying the number of primary panicle branches and/or the number of formed grains on a plant is provided. The chemical agent may include, as the gene serving as the active ingredient, the foregoing vector.

Transformed Plant

In the transformed plant disclosed herein, expression by the gene which regulates the number of primary panicle branches is enhanced. The primary panicle branch number-regulating gene which is enhanced may be a gene endogenous to the plant, or may be an exogenous gene. Or the gene may be both endogenous and exogenous. When gene expression is “enhanced,” this means that the level of expression by the gene (the amount of primary transcript by the gene, or the amount of protein coded for by the gene that is produced) is increased compared to before transformation or that the activity of the protein is increased compared to before transformation. As a result of enhanced expression of the gene, the activity itself of the protein may increase together with the increase in the level of expression by the gene.

No particular limitation is imposed on the form in which expression by the gene is enhanced. In one exemplary form, a promoter which is operative in the plant cell and the gene which is operatively linked with the promoter are carried as exogenous DNA, either on a chromosome of the plant cell or extrachromosomally. The gene which is linked to the promoter may be one that is endogenous to the plant cell, or one that is exogenous. Further examples include, in order to increase the activity of the promoter for the gene when it is endogenous, a form which involves substituting all or part of the promoter region on the chromosome, and a form which involves substituting the promoter region together with the endogenous gene.

The transformed plant of the invention includes a plant cell into which has been inserted the vector disclosed herein intended for transferring the gene into the plant cell and induce expression.

The transformed plant of the invention may be obtained by regenerating the plant from a plant cell transformed by insertion of the vector disclosed herein.

Insertion of the vector into a plant cell may be carried out using any of various techniques known to persons skilled in the art, such as the polyethylene glycol method, electroporation, the Agrobacterium-mediated method, and the particle gun method. Specific examples include gene transfer to a protoplast by polyethylene glycol (Datta, S. K., in Gene Transfer to Plants, edited by Potrykus, I. and Spangenberg (1995), pp. 66-74); gene transfer to a protoplast by electrical pulses (Told et al., Plant Physiol. 100, 1503-1507 (1992)); direct injection of a gene into a cell by the particle gun method (Christou et al., Bio/Technology, 9:957-962 (1991)); and Agrobacterium-mediated gene transfer (Hiei et al., Plant J., 6:271-282 (1994)). Regeneration of a plant from the transformed cell may be carried out by a method known to persons skilled in the art, depending on the type of plant cell (see Told et al., Plant Physiol. 100:1503-1507 (1995)). Examples of such methods include, for rice, the method of Fujimura et al. (Plant Tissue Culture Lett., 2:74 (1995)); for corn, the method of Shillito et al. (Bio/Technology, 7:581 (1989)) and the method of Gorden-Kamm et al. (Plant Cell 2:603 (1990)); for potato, the method of Visser et al. (Theor. Appl. Genet., 78:594 (1989)); for tobacco, the method of Nagata and Takebe (Planta 99:12 (1971)), and for A. thaliana, the method of Akama et al. (Plant Cell Reports, 12:7-11 (1992)).

Regeneration of a plant from the transformed cell may be carried out by a method known to persons skilled in the art, depending on the type of plant cell (see Toki et al., Plant Physiol. 100:1503-1507 (1995)). For example, in the case of rice, several techniques have already been established and are in wide use in the technical field of the invention as techniques for producing transformed plants, including a method involving gene transfer to a protoplast by polyethylene glycol, followed by regeneration of the plant (Indica rice varieties are suitable) (Datta, S. K., in Gene Transfer to Plants (edited by Potrykus I. and Spangenberg), (1995), pp. 66-74)); a method involving gene transfer to a protoplast by electrical pulses followed by regeneration of the plant (Japonica rice varieties are suitable) (Toki et al., Plant Physio. 100, 1503-1507 (1992)); a method involving direct injection of a gene into a cell by the particle gun method, followed by regeneration of the plant (Christou et al., Bio/Technology, 9:957-62 (1991)); and a method involving Agrobacterium-mediated gene transfer, followed by regeneration of the plant (Hiei et al., Plant J., 6:271-282 (1994)). Preferred use may be made of these methods in the present invention.

If a transformed plant in which the gene has been integrated onto the genome can be obtained, it is possible to obtain progeny from the plant by sexual or asexual reproduction. It is also possible to obtain a propagation material from the plant or a progeny or clone thereof (examples of propagation materials include seed, fruit, cut panicles, tubers, root tubers, stocks, calluses, protoplasts), and to mass-produce the plant based on these. The present teaching includes the following which have already been described: (1) plant cells into which the gene has been transferred, (2) plants containing such cells, (3) the progeny and clones of such plants, and (4) propagation materials from such plants or the progeny and clones thereof.

Because the plant thus produced has been conferred with a primary panicle branch number-increasing ability or such an ability has been enhanced therein, the plant has an increased number of formed grains or an increased foliage yield.

The present teaching provides a polynucleotide having the base sequence set forth in SEQ ID NO:1 or containing at least 15 consecutive bases which are complementary to a complementary sequence thereto. Here, “complementary sequence” refers to, with respect to the sequence of the one strand of double-stranded DNA composed of A:T and G:C base pairs, the sequence of the other strand. Also, the term ‘complementary’ is not limited to cases in which the complementary sequence is perfect in the region of at least 15 consecutive nucleotides, and may refer to a base sequence identity of at least 70%, preferably at least 80%, more preferably 90%, still more preferably at least 95%, and even more preferably at least 95%. Such DNA is useful as a probe for carrying out the detection and isolation of the gene, or as a primer for carrying out gene amplification.

Method of Determining the Number of Primary Panicle Branches in a Plant

The present teaching provides a method for determining changes in the number of primary panicle branches in a plant. That is, the teaching provides a method for determining the number of primary panicle branches in a plant, which method is characterized by including the step of analyzing expression of the gene in the plant or a portion thereof. Such gene expression analysis can be carried out by a method familiar to persons skilled in the art. For example, by preparing an RNA specimen containing RNA from a test plant or propagation material thereof and using reverse transcriptase to synthesize cDNA from RNA in the specimen, the level of expression can be evaluated based on the amount of eDNA synthesized. By way of illustration, in eases where the level of expression by the gene obtained in expression analysis is lower than that of the housekeeping genes or the OsSPL14 gene in NP-12, the test plant can be judged to have a low number of primary panicle branches or to have a primary panicle branch number which is suppressed. In cases where the degree of expression is equivalent to or higher than that of the housekeeping genes or the OsSPL14 gene in NP-12, the test plant can be judged to have a high number of primary panicle branches or to have a primary panicle branch number which is enhanced.

A known expression analysis technique, such as a DNA microarray using the above-described probe and primer, or real-time PCR, may be suitably used for expression analysis. The gene tends in particular to be specifically expressed in panicles. In particular, in relatively small panicles (typically ones smaller than 10 mm, more preferably smaller than 5 mm, and even more preferably smaller than 2 mm), the level of expression has a tendency to be high. Accordingly, it is possible, even by expression analysis of this portion, to make a determination on changes in the number of primary panicle branches.

As used herein, the phrase “determination on changes in the number of primary panicle branches” refers not only to determinations on changes in the number of formed grains in varieties which have been cultivated to date, but includes also determinations on changes in the number of formed grains in new varieties that arise due to crossing and genetic recombination.

This method of determination provides advantages in, for example, cases where breeding is carried out by crossing plants. Fox example, in cases where the introduction of a trait which increases the number of primary panicle branches is not desired, such as when the aim is to lower the number of formed grains, it is possible to avoid crossing plants having characteristics that increase primary panicle branches, or, conversely, in cases where the introduction of a trait which increases the number of primary panicle branches is desired, such as when the aim is to increase the number of formed grains, crossing with a variety having the characteristic of increasing the number of primary panicle branches may be carried out. This is also effective when screening for desirable individuals from the progeny of a cross. Because a change in the number of primary panicle branches is simpler and more reliable to judge at the gene level than to judge based on the phenotype, this method of determination is capable of making significant contributions to plant breeding.

Method of Producing a Useful Crop

The method of producing a useful crop disclosed herein includes the step of cultivating the transformed plant, and the step of harvesting the transformed plant or a portion thereof. This production method enables a crop having high number of formed grains and a high yield of foliage to be obtained, and enables more seed and foliage to be harvested. Both the cultivating step and the harvesting step may be suitably set according to the type of transformed plant. In this production method, in cases where the transformed plant is a plant in which the seeds serve as a useful portion, such as gramineous plants wherein the seeds form into grains, a larger quantity of seed can be harvested. At the same time, the straw can also be harvested. In cases where the transformed plant is one in which the foliage serves as a useful portion, a larger amount of foliage can be harvested.

Method of Regulating Yield

This specification also discloses a method for regulating the yield of a plant or a portion thereof, which method is characterized by regulating the expression of the gene in the plant. The method of regulation disclosed herein is able, by enhancing expression of the gene, to increase the number of primary panicle branches and to increase the yield of seeds or foliage. This method is also able, by suppressing expression of the gene, to reduce the number of primary panicle branches. Expression of the gene in the plant can be enhanced by, for example, as already explained above, producing a transformed plant by using the vector disclosed in this specification. Expression of the gene within the plant can be suppressed by using, on the gene endogenous to the plant, a method for suppressing gene expression in plants which is commonly known to persons skilled in the art, such as an antisense, ribozyme, cosuppression or dominant negative method.

Other Forms

The Plant

Another form of the plant disclosed in this specification is a plant which carries a DNA region, the region including a first DNA encoding the protein and, upstream of the first DNA, a second DNA mentioned in (g) to (j) below, at an original chromosomal locus of the first DNA or at a position corresponding to this locus:
(g) DNA having the base sequence set forth in SEQ ID NO:3;
(h) DNA which has a base sequence having, in the base sequence set forth in SEQ ID NO:3, one or more substituted, deleted, added andior inserted base, and which has an ability to enhance expression of a protein having a primary panicle branch number-increasing activity;
(i) DNA which hybridizes under stringent conditions with a complementary strand of DNA having the base sequence set forth in SEQ ID NO:3, and which has an ability to enhance expression of a protein having a primary panicle branch number-increasing activity;
(j) DNA which has an identity of at least 70% (preferably at least 75%, more preferably at least 80%, even more preferably at least 85%, still more preferably at least 90%, still yet more preferably at least 95%, even more preferably at least 98%, and most preferably at least 99%) with the base sequence set forth in SEQ ID NO:3, and which has an ability to enhance the expression of a protein having a primary panicle branch number-increasing activity.

In the DNA of (h) above, no particular limitations are imposed on the number and types of base changes (substitutions, deletions, additions and/or insertions). Also, the identity in the DNA of (j) above has already been described. The “protein having a primary panicle branch number-increasing activity” is the protein which has already been described above. The DNA of (h) to (j) above is exemplified by, when the level of expression of the protein is observed to increase at the stage of development in a plant such as rice which has a large number of primary panicle branches, like NP-12, DNA which exists on the upstream side of the protein on a chromosome of the plant. Moreover, in above (h) to (j), it is preferable for the DNA to have no substitutions or other changes at the subsequently described sites of single base polymorphism included in the base sequence set forth in SEQ ID NO:3.

The inventors have discovered that the gene participates in an increase in the primary particle branch number in NP-12. In addition, they have found that a 2.6 kb region upstream thereto plays a role in enhancing expression of the gene. In NP-12, this 2.6 kb region itself is the same as the corresponding region in Nipponbare, a Japonica variety of rice. However, in NP-12, the upstream and downstream sides of this 2.6 kb, region have a total of five single base substitutions (single base polymorphisms). The reason for the increase in the number of primary panicle branches in NP-12 is thought to be due to a higher level of expression of the gene during the growth stage of the plant. Therefore, by providing this upstream region from NP-12, that is, a region containing at least the 2.6 kb region and the regions containing two single base substitutions adjoining both ends thereof, on the upstream side of the gene on a chromosome of plants such as a gramineous plant other than NP-12, an increase in the number of primary panicle branches or an increase in yield like that in NP-12 can be achieved. Moreover, it is also possible to provide, in an upstream region of the gene, a region which contains a total of five single base substitutions, including three single base substitutions in addition to the two above single base substitutions at or near the above 2.6 kb region.

The base sequence set forth in. SEQ ID NO:3 defining the second DNA is a region from NP-12 which includes the above 2.6 kb region and has one base adjacent to the 5′ end thereof and one base adjacent the 3′ end thereof. This is a base sequence consisting of 2,593 base pairs made up of the 2.6 kb region (the base sequence of 2,591 base pairs set forth in SEQ ID NO:4) and additionally has a single base substitution (C→T) at position 2 and a single base substitution (G→A) at position 3 on the 5′ end.

In addition, the second DNA may be DNA consisting of a base sequence of about 4 kb (SEQ ID NO:5) which includes the above 2.6 kb and includes all the single base polymorphisms. The 2.6 kb region corresponds to positions 30 to 2620 on the base sequence set forth in SEQ ID NO:5. Although it is not entirely clear what role the five single base substitutions play in this 2.6 kb region, the single base polymorphisms at positions 2 and 3 which are closest to the 2.6 kb region are thought to take part in increasing the number of primary panicle branches in NP-12.

The single base substitution sites in the base sequence set forth in SEQ ID NO:5 are as shown below. The bases prior to substitution were obtained from the alignment results using the BLAST program on the sequence reported for Oryza sativa, Japonica Group DNA, chromosome 8, complete sequence, cultivar, Nipponbare (accession No.: NC_{—}008401.2), and the sequence from NP-12.
(1) 1: C→T
(2) 29: C→T
(3) 2621: G→A
(4) 3474: C→T
(5) 3827: C→T

Alternatively, the second DNA may be any of the following DNA. In the DNA of (l) to (n) below, it is preferable for there to be no substitutions or other changes at the five sites of single base polymorphism included in the base sequence set forth in SEQ ID NO:5.
(k) DNA composed of the base sequence set forth in SEQ ID NO:5;
(l) DNA which has a base sequence having, in the base sequence set forth in SEQ ID NO:5, one or more substituted, deleted, added and/or inserted base, and which has an ability to enhance expression of a protein having a primary particle branch number-increasing activity;
(m) DNA which hybridizes under stringent conditions with a complementary strand of DNA having the base sequence set forth in SEQ ID NO:5, and which has an ability to enhance expression of a protein having a primary panicle branch number-increasing activity;
(n) DNA which has an identity of at least 70% (preferably at least 75%, more preferably at least 80%, even more preferably at least 85%, still more preferably at least 90%, still yet more preferably at least 95%, even more preferably at least 98%, and most preferably at least 99%) with the base sequence set forth in SEQ ID NO:5, and which has an ability to enhance expression of a protein having a primary panicle branch number-increasing activity.

A DNA region containing the second DNA and also containing the first DNA is exemplified by DNA composed of the base sequences set forth in SEQ ID NO:6 and SEQ ID NO:7.

The plant of the invention carries such a DNA region at an original chromosomal locus of the gene, or at a position corresponding to the locus. Here, “original chromosomal locus of the gene” refers to, in cases where a plant originally bears the gene or a homolog thereof, the chromosomal locus where the gene is endogenous. “Position corresponding to the original chromosomal locus” pertains to cases where, although the position does not perfectly match the locus, the DNA region is positioned near enough to the locus from the base sequence thereabout as to not interfere with the gene expression-enhancing activity of the second DNA. The plant is preferably a plant which already bears the gene as an endogenous gene on a chromosome. For example, in rice, the gene locus for the OsSPL14 gene and its homolog is chromosome No. 8. The gene, that is, the OsSPL14 gene in rice, is known to be common to, and present in, sorghum, wheat, corn and A. thaliana.

Whether the plant bears such a DNA region can be ascertained by detecting the presence/absence, length and the like of products of amplification using PCR on a DNA region containing the base sequence of the second DNA. Whether such a DNA region is positioned at this gene locus can be determined by a known base sequencing technique.

The plant of the invention may be a plant which already exists as a pure-bred variety, such as NP-12, or may be a plant other than NP-12 which is a hybrid obtained by crossing with NP-12 or the progeny of such a hybrid. The progeny may be a pure-bred variety established by cross-breeding. Alternatively, the plant may be a transformant obtained by breeding by genetic manipulation, or may be the progeny of such a transformant. Such progeny may be plants obtained by cross-breeding from transformants.

Other than methods which involve gene recombination and gene targeting, the plant of the invention may also be acquired by crossing. When crossing is used, an F1 generation carrying the above DNA region at the original chromosomal position of the gene or at a position corresponding to that position may be obtained by homologous recombination during fertilization between, for example, NP-12 and another plant. A homozygote for this DNA region (allele) can be obtained by making use of, for example, the F2 generation or by back-crossing. The plant of the invention may, at the locus for the gene, be heterozygous for the allele containing this DNA region, although it is preferably homozygous.

The plant of the invention may be a monocotyledon, of which rice is an example. The plant is preferably rice or another gramineous plant other than rice (examples of which include wheat, barley, corn, A. thaliana and sorghum).

The plant of the invention is in itself useful as a plant which has a high number of primary panicle branches, or as a plant having a high yield, either exclusive of the seeds or including the seeds. Moreover, this plant is also useful as a new variety for breeding.

Method of Producing the Plant

The method of producing a plant disclosed in the specification includes the step of crossing a parental variety of plant which carries a DNA region, the region including a first DNA encoding any one of proteins (a) to (f) above and, upstream of the first DNA, a second DNA (g) to (j) above, at a locus where the first DNA is originally positioned or at a position corresponding to this locus with other plant so as to produce a new variety of plant which carries this DNA region at the locus where the first DNA is originally positioned or at a position corresponding to this locus. A plant having an increased number of primary panicle branches or an increased yield can be obtained by this method of production. The parental variety of plant may be NP-12 or other plants of a similar form. The other plant is preferably a plant which does not contain the second DNA, but which contains the first DNA, i.e., the gene. The other plant is preferably a monocotyledon, and more preferably a gramineous plant. It is even more preferably rice.

The crossing of the parental variety of plant with other plant may be carried out by a person of ordinary skill in the art using a technique that is already known. Plants obtained by crossing may be screened for by detecting one, two or more polymorphisms selected from the five single base polymorphisms contained in the second DNA. That is, at least part of the base sequence of the second DNA, and more precisely a region containing one, two or more polymorphisms selected from among the five single base polymorphisms included in the base sequence, may be used as a good marker for breeding plants having a large number of primary panicle branches and a large number of formed grains. DNA composed of a base sequence containing at least one of the five single base polymorphisms in the base sequences set forth in SEQ ID NO:3, SEQ ID NO:5, SEQ ID NO:6 and SEQ ID NO:7 may be used as this region.

The detection of single base polymorphisrn.s may be suitably carried out by, for example, PCR, hybridization, sequencing, or combinations of these techniques. The primers and probes may be suitably selected according to the sequence set forth in any of SEQ ID NO:3 to SEQ ID NO:7.

The present teaching of the specification also discloses a DNA fragment for breeding which has a DNA region that includes the first DNA and, upstream of the first DNA, the second DNA. Such a DNA fragment, i.e., an OsSPL14 allele having the base sequence characteristics in NP-12, is an important breeding allele (DNA fragment) which plays a part in increasing the number of primary panicle branches or the number of formed grains on a plant.

The present teaching of the specification farther discloses a DNA fragment for breeding which includes this second DNA. The second DNA itself acts on the OsSPL14 gene of NP-12 which has the same base sequence as in Nipponbare, contributing to an increase in the number of primary panicle branches and the number of formed grains. Therefore, this DNA fragment itself may be regarded as a DNA fragment useful for breeding.

The present teaching of the specification discloses the use of the above DNA region, such as the DNA region on the chromosome No. 8 of the NP-12 stock strain, of rice preserved at Nagoya University, in the production of a plant of another form having an increased number of primary panicle branches or an increased yield. Accordingly, this specification discloses a method of producing a plant which includes the step of cultivating such a plant of another form, and the step of harvesting this plant of another form or a portion thereof.

### Examples

The invention is illustrated more fully below by way of examples, although these examples are not intended to limit the scope of the invention.

### Example 1

### Identification of Gene Regulating Number of Primary Panicle Branches

The Japonica variety of rice Nipponbare as a cultivated variety and the Indica variety of rice NP-12 (a strain preserved at Nagoya University) as a high-yield variety, both differing clearly in their respective number of primary panicle branches, were selected for use as the parents of a hybrid population to be subjected to QTL analysis (FIG. 1). The plants were incubated in water within a Petri dish at 30° C. for 72 hours to effect germination, following which they were transplanted into pots having a diameter of 10 cm and a height of 13 cm. The number of primary particle branches was measured after the grains had ripened.

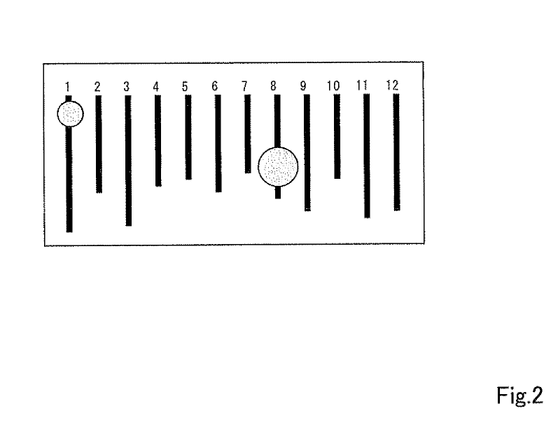

A QTL analysis was carried out on an F2 population of 3,200 plants obtained by self-propagation of F1 individuals produced by crossing Nipponbare with NP-12. As shown in FIG. 2, loci regulating the number of primary panicle branches were detected on the short arm of chromosome No. 1 and the long arm of chromosome No. 8. Of these QTLs, the primary panicle branch number-increasing effects were found to be greater on the long arm of chromosome No. 8.

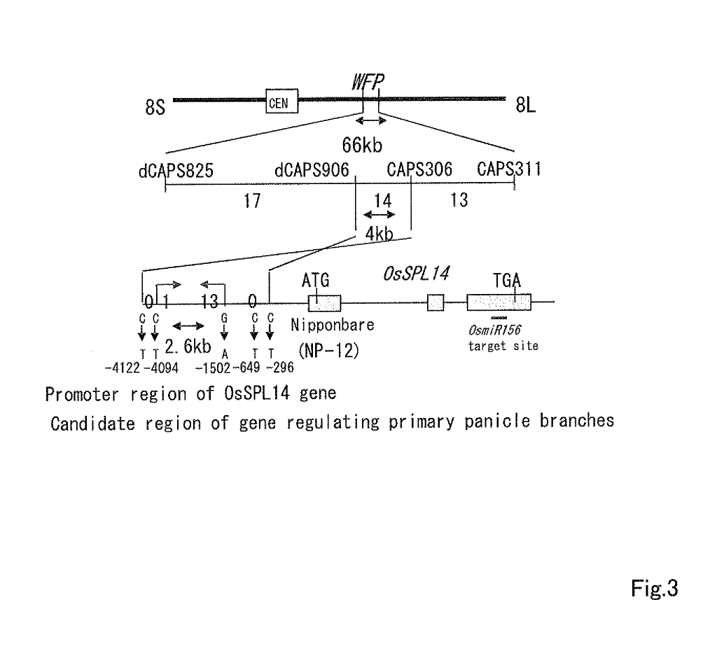

Using F3 generation progeny of the F2 population, a positional cloning method was carried out in order to specify the QTL on chromosome No. 8. The results are shown in FIG. 3.

As shown in FIG. 3, it was possible to identify the candidate region for the Wealthy farmer's panicle (WFP) gene, a gene in NP-12 which increases the number of primary panicle branches, on a 2.6 kb region upstream of the OsSPL14 gene. Because the WFP gene candidate region was identified on the region upstream of the OsSPL14 gene, it was conjectured that changes to this region in NP-12 were altering the level of expression of the OsSPL14 gene.

As shown in FIG. 1, upstream and downstream from this 2.6 kb region, there were five single base substitutions with respect to the same base sequence in Nipponbare. The values marked at each of the substitution sites indicate the position from the first base on the mRNA (Accession No.: NM_{—}001068739) of 0s08g0509600 of Nipponbare.

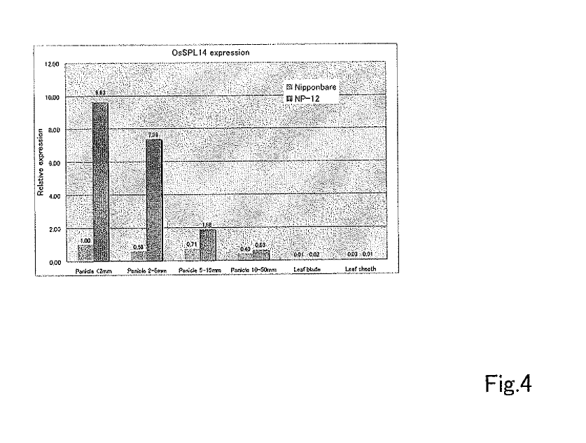

Next, the levels of expression of OsSPL14 in Nipponbare and NP-12 at each stage of panicle development were compared. The results are shown in FIG. 4. Expression analysis was carried out by using Trizol (invitrogen) to extract RNA, and using Omniscript (QIAGEN) to synthesize cDNA. Using a CYBR Green RT-PCR kit (QIAGEN) as a fluorescent reagent and using Light Cycler (Roche) as the detector, a PCR reaction was carried out on this cDNA, and the level of expression was quantitatively determined using OsUbiquifin as the internal standard gene.

As shown in FIG. 4, the level of expression at the start of panicle development was very high in NP-12 compared with Nipponbare. From these results, the gene that increases the number of primary panicle branches in the high-yielding rice NP-12, i.e., the WFP gene, was surmised to be the causative gene as a high-expression allele of the OsSPL14 gene owing to an upstream mutation in the OsSPL14 gene.

### Example 2

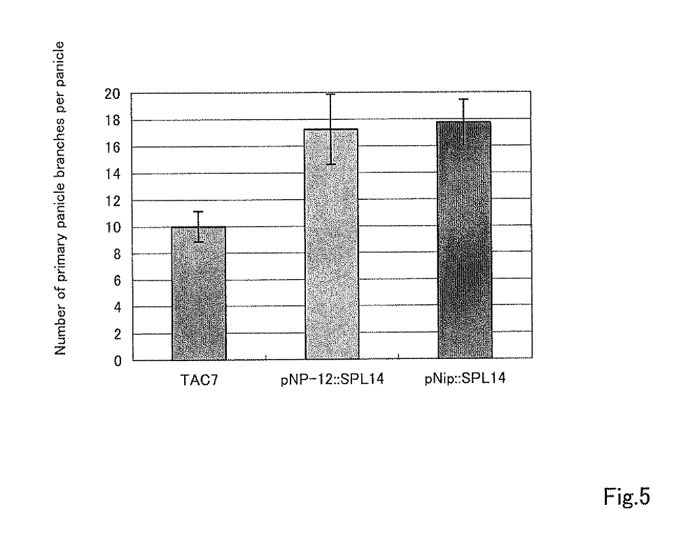

To corroborate the above, an attempt was made to transfer the OsSPL14 gene from NP-12 into Nipponbare. That is, a transforming plasmid was constructed as described below, and transformation was carried out. The number of primary panicle branches in the resulting transformed plant was then determined. The results are shown in FIG. 5.

Construction of Plasmid and Transformation of Plant

In order to create a transformant to which the OsSPL14 gene of NP-12 has been transferred, OsSPL14 was isolated and transferred to the binary vector pYLTAC7 (supplied by RIKEN). This binary vector was transferred to Agrobacterium EHA105 strain by electroporation. Rice was transformed by a method described in the literature (Hiei, Y., Ohta, S., Komari, T. & Kumashiro, T.: “Efficient transformation of rice (Oryza sativa L.) mediated by Agrobacterium and sequence analysis of the boundaries of the T-DNA,” Plant J., 6, 271-282 (1994)). Briefly, this involved infecting a Nipponbare callus with, and thereby introducing, Agrobacterium EHAI 05 into which a DNA fragment had been transferred, and subsequently screening for the transformed plants in a medium containing 50 mg/L of hygromycin. The hygromycin-tolerant plants were transplanted into soil, and cultivated under the following conditions: 30°C, 16 hours light/8 hours dark per day.

As shown in FIG. 5, individuals transformed with the TAC7 vector alone formed about ten primary panicle branches, whereas the individuals into which the OsSPL14 gene from NP-12 had been transferred (NP-12::SPL14) formed about 17 primary panicle branches. From these results, it was concluded that the OsSPL14 gene is the gene WFP which increases the number of primary panicle branches. In addition, the number of primary panicle branches also increased in the individuals into which the OsSPL14 gene from Nipponbare had been transferred (Nip::SPL14). From these results, it appeared that the gene effects of the OsSPL14 gene are enhanced even when the number of copies of the gene increases.

### Example 3

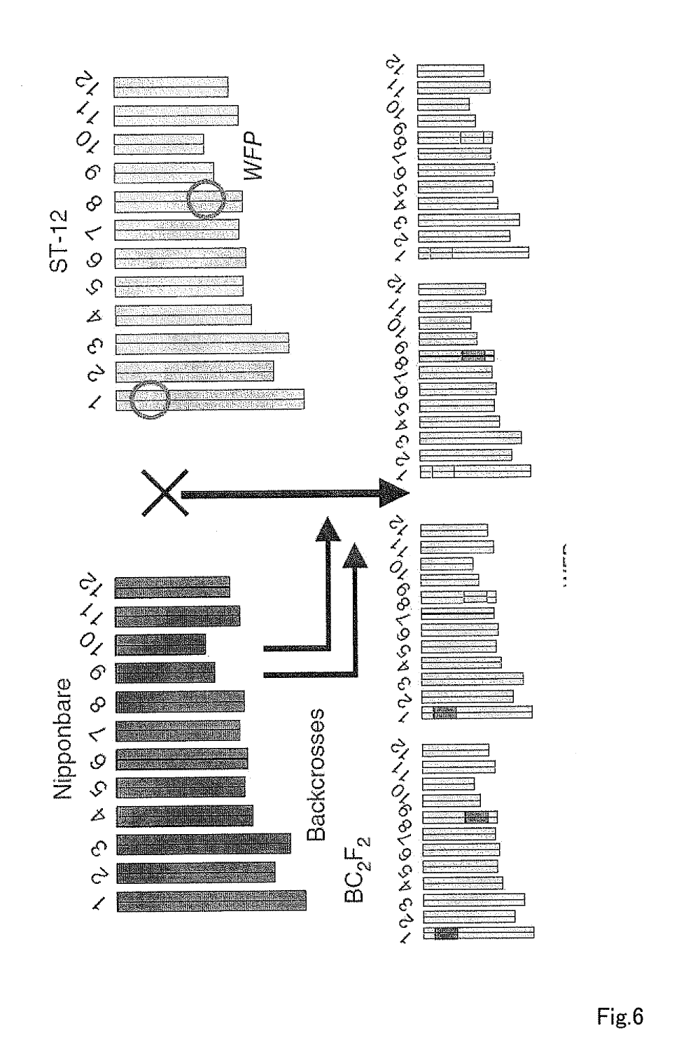

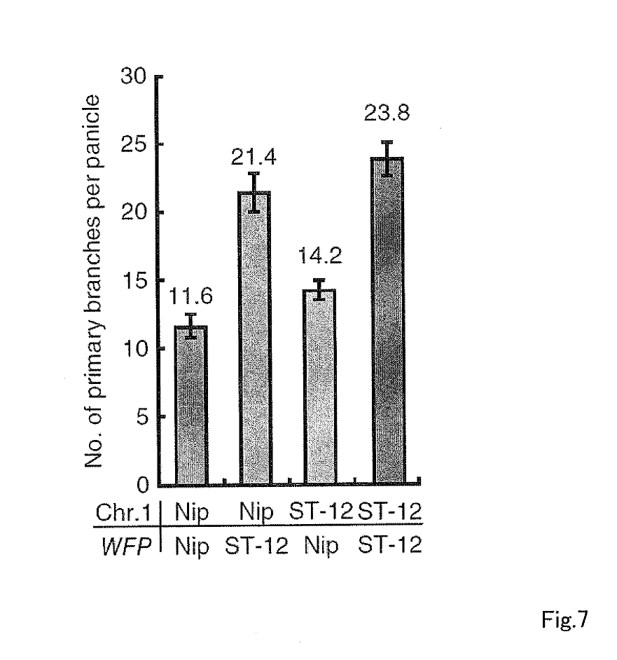

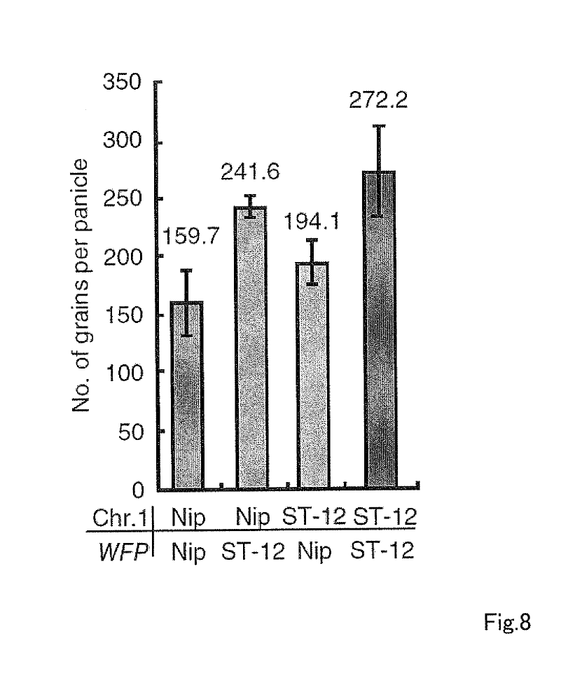

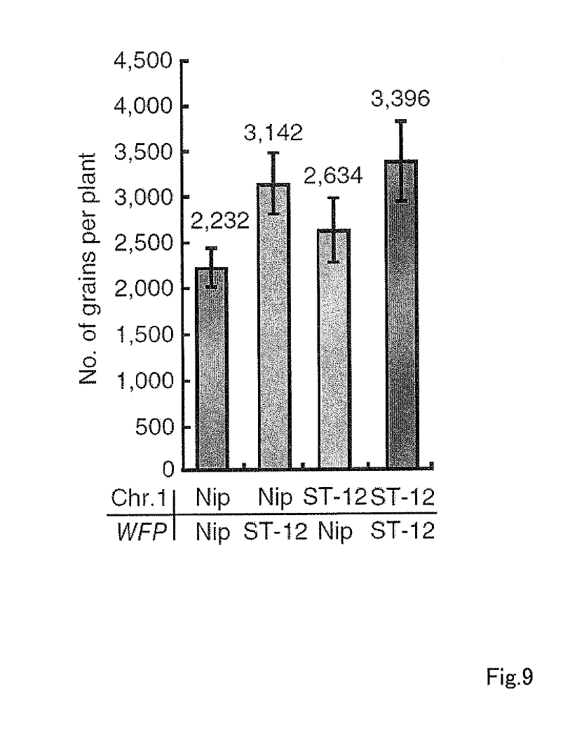

In this example, the effects by the QTL on chromosome No. 8 (2.6 kb region) and by the QTL on chromosome No. 1 on the amount of harvested grain were evaluated. The genotypes of four kinds of BC₂F₂ progeny obtained by crossing Nipponbare with NP-12, then back-crossing twice with Nipponbare are shown in FIG. 6. The QTL on chromosome No. 1 and the QTL on chromosome No. 8, both from NP-12, are indicated by the red circles. In this example, the QTL on chromosome No. 8 included a 52.6 kb region; within this region, a region containing five single base substitutions was used as a marker. The number of primary branches per main panicle was measured for these four types of plants, as a result of which the number of grains per main panicle and the number of grains per plant were also obtained. These results are shown respectively in FIGS. 7, 8 and 9. Forty individuals were used for each of the four types of plants.

As shown in FIGS. 7 to 9, the OsSPL14 gene on chromosome No. 8 played a role in an increase of about 40% in the number of primary branches and the number of grains. In addition, in plants having the QTL on chromosome No. 1 from NP-12 and having OsSPL14 on chromosome No. 8, the number of primary branches per main panicle was 23.8, the number of grains per main panicle was 272.2, and the total number of grains per plant was 3,396. In other words, the number of primary branches per panicle increased by 12.2 relative to the 11.8 primary branches per particle in plants wherein both QTLs were from Nipponbare. Moreover, the total number of grains per plant increased by 1,164 (54%) relative to the total of 2,232 grains per plant in the plants wherein both QTLs were from Nipponbare.

Hence, the OsSPL14 allele on chromosome No. 8 from NP-12 had a strong grain number-increasing effect. Moreover, it was apparent from the above results that the base sequence (SEQ ID NO:3) containing a 2.6 kb region and two single base substitutions adjacent to the end of this 2.6 kb region has a strong grain number-increasing effect, identifying it as a useful allele for breeding, and is also a useful marker.

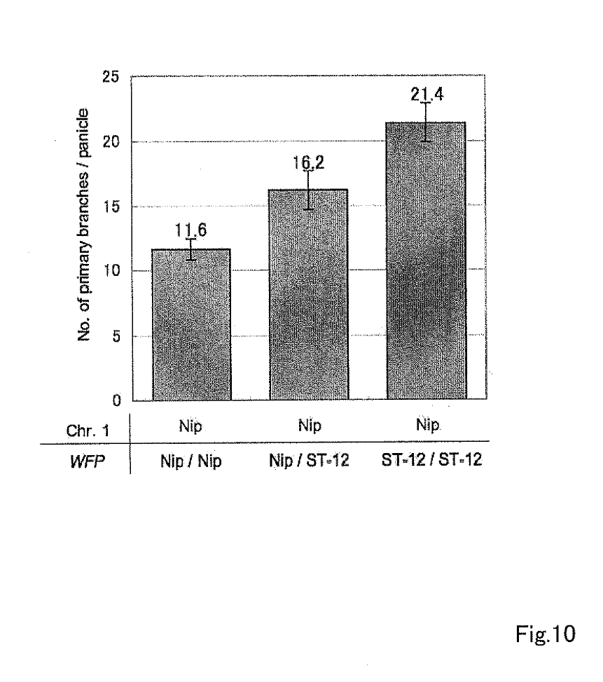

In addition, as shown in FIG. 10, with regard to a heterozygote of Nipponbare and NP-12 for the QTL on chromosome No, 8, when the number of primary branches per main panicle was evaluated, the number of primary branches in this heterozygote was found to be a value intermediate between those for the two homozygotes for the QTL on chromosome No. 8. This indicated that the QTL from NP-12 on chromosome No. 8 (the OsSPL14 allele) was semidominant.

### Example 4

When this 2.6 kb sequence in Nipponbare and NP-12 were compared, no disparity whatsoever could be found. Expression analysis of this region was carried out, but expression was detected in neither Nipponbare nor NP-12. Nor was it possible, even in databases, to find any evidence of the transcription of these in either plant.

Inherited disparities in gene expression which do not depend on changes in the DNA sequence are defined as epigenetic alleles. Epigenetic alleles have been reported in A. thaliana and rice. In order to determine whether inherited epigenetic marks in endogenous OsSPL14 promoters play a role in OsSPL14 level of expression disparities, the methylation levels were evaluated by bisulfite sequence analysis of this region. Cytosines on single-stranded DNA were sulfonated with bisulfite (sodium bisulfate), and hydrodeamination was carried out, followed by desulfonation, thereby converting the cytosines to uracil (U). At the same time, because methylated cytosine remains unchanged as methylated cytosine, by carrying out PCR amplification using the bisulfite-treated DNA as the template and reading the base sequence, methylated cytosine sites (C) and non-methylated cytosine sites (T) can be distinguished from each other during reading.

Bisulfite sequence analysis was performed by using the genomic DNA extracted from Nipponbare and NP-12 and an EpiTect Bisulfite Kit (QIAGEN 59104) to carry out bisulfite treatment. The 2.6 kb region was amplified using bisulfite-converted primers, and cloned to a pCr-4 vector. The base sequence of the amplification product was analyzed for at least 24 clones. The sequences of the bisulfite-converted primers used are shown below.

TABLE 1

| | Forward (5′→3′) | Reverse (5′→3′) |
|---|---|---|
| Bis-bottom-4 | CCGTATTAACCTCGTGCCGTAACCATCTTA | TCGTACACATATAACGTTTTGGAGTCTGTG |
| Bis-bottom-5 | ACCCTGCCACATACTACTCTACGCCAAAAT | ACACATTCACTATTGCTTTGGTAGAAGTTA |
| Bis-bottom-6 | GCTCCTCCATCGGTAGCAGCACACTATTCC | GTGGGCTCCGAACGAAGGGTGAATAGTTAT |
| Ms-bottom-7 | TTCATCTCAACATCCTTTCCTCTTCTACTT | TTAAAATGTGTAGTTTTATGAGAATGGAGA |
| Bis-top-4 | GCAATAGTGAATGTGTACCATGGAGAGAAG | AGCTTACTATTATAGCTAGCCAATCTAATA |
| Bis-top-5 | TGTTGTGCTGATGGATAAGAGG | CTACTACTTCGTCGAGCTCTCATCAAT |
| Bis-top-6 | ATAGCTTCTGCGTGATTTGATAACTGGAGG | ACCGTCCTTGCCCTCTCATAACTATTCTCA |
| Bis-top-7 | TATATTAATGGTGTAGTATATGTTTATAAGCA | TTCAATATCTCCATTCTCATAAAACTATAC |

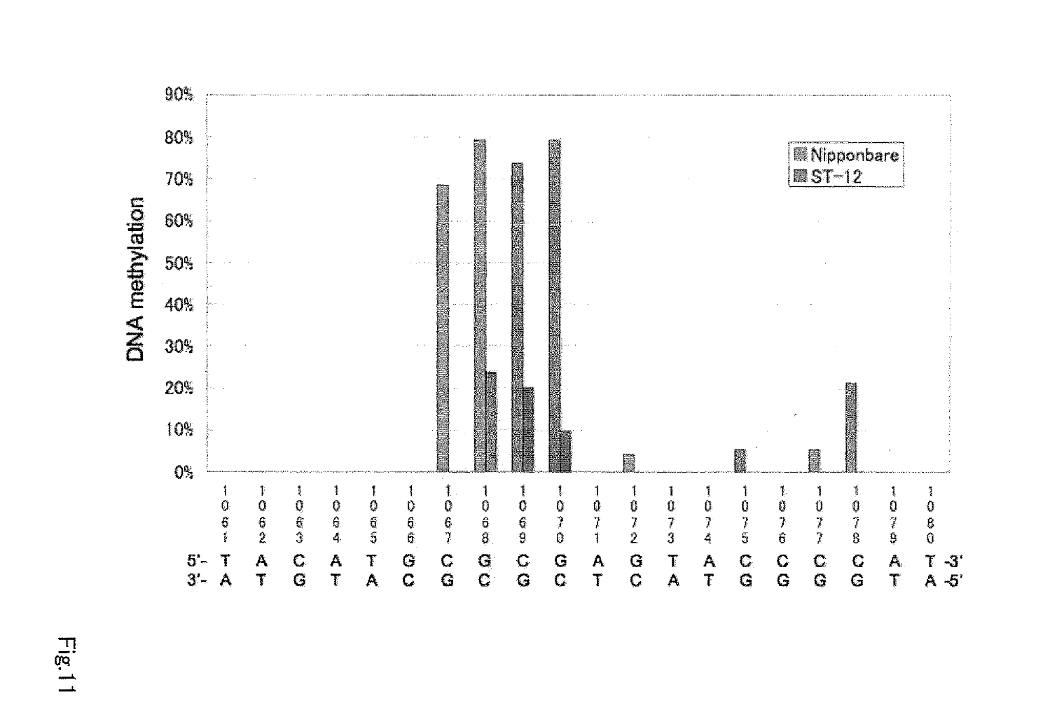

The results showed that there was no large difference between Nipponbare and NP-12 in the DNA methylation levels for the 2.6 kb region as a whole. Yet, as shown in FIG. 11, within the 2.6 kb region, there were disparities between the two in the methylation levels at several cytosines near base 1070. At these cytosines, the methylation levels in NP-12 were between 0 and 24%, whereas higher methylation levels of between 68 and 79% were observed in Nipponbare.

1-18. (canceled)

19. A vector, which carries a DNA region including a first DNA encoding any of proteins (a) to (f) below and, upstream of the first DNA, a promoter region of SPL14 gene of NP-12, a stock strain of rice preserved at Nagoya University:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a. protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity.

20. A plant cell to which the vector according to claim 19 has been transferred.

21. A transformed plant containing the pant cell according to claim 20.

22. A transformed plant which is a plant or clone of transformed plant according to claim 21.

23. A propagation material of the transformed plant according to claim 21.

24. A plant, which has is obtained by crossing NP-12, a stock strain of rice preserved at Nagoya University with other plant and has an increased number of primary panicle branches.

25. A propagation material of the plant according to claim 24.

26. A method of producing a transformed plant, the method comprises:
transferring in use of the vector according to claim 19 said gene into a plant cell and regenerating a plant from the plant cell.

27. A method of producing a plant, the method comprises:
crossing NP-12, a stock strain of rice preserved at Nagoya University with other plant, and
cultivating a propagation material of a plant obtained by the crossing and producing the plant having an increased number of primary panicle branches.

28. A method of producing a useful crop, the method comprises:
cultivating the transformed plant according to claim 21, and
harvesting the plant or a portion thereof.

29. A method of producing a useful crop, the method comprises:
cultivating the plant according to claim 24, and
harvesting the plant or a portion thereof.

30. A method of regulating a yield of a plant or a portion thereof, the method comprises:
regulating, by use of a promoter region of SPL gene of NP-12, a stock strain of rice preserved at Nagoya University, a level of expression of a gene encoding any one of proteins (a) to (f) below in the plant:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity.

31. A chemical agent for modifying a yield of a plant or a portion thereof, the chemical agent comprises, as an active ingredient, a DNA encoding one of proteins (a) to (f) below:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity.

32. A plant, the plant carries a DNA region including a first DNA encoding any of proteins (a) to (f) below and, upstream of the first DNA, a second DNA (g) below, at a locus where the first DNA is originally positioned or at a position corresponding to said locus:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO: 1, and which has a primary panicle branch number-increasing activity;
(f) a protein which is encoded by DNA having at least 70% identity with the base sequence set forth in SEQ ID NO:1, and which has a primary panicle branch number-increasing activity.
(g) DNA which has a base sequence set forth in SEQ ID NO:3.

33. The plant according to claim 32, wherein the plant is a monocotyledon.

34. The plant according to claim 33, wherein the monocotyledon is a gramineous plant.

35. A propagation material of the plant according to claim 32.

36. A method of producing a plant, the method comprises crossing an parental variety of plant which carries a DNA region, including a first DNA encoding any of proteins (a) to (f) below and, upstream of the first DNA, a second DNA (g) below, at a locus where the first DNA is originally positioned or at a position corresponding to said locus with other plant so as to produce a new variety of plant which carries said DNA region at said locus where the first DNA is originally positioned or at a position corresponding to said locus:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity
(g) DNA which has a base sequence set forth in SEQ ID NO:3.

37. The production method according to claim 36, the method further comprises screening the new variety of plant by using, as a marker, DNA containing at least a portion of the second DNA.

38. A vector , which carries a DNA region, including a first DNA encoding any of proteins (a) to (f) below and, upstream of the first DNA, a second DNA (g) below:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity;
(g) DNA which has a base sequence set forth in SEQ ID NO:3.

39. A plant cell to which the vector according to claim 38 has been transferred.

40. A transformed plant containing the plant cell according to claim 39.

41. A transformed plant which is a progeny or clone of the transformed plant according to claim 40.

42. A propagation material for the transformed plant according to claim 40.

43. A method of producing a transformed plant, the method comprises transferring in use of the vector according to claim 38 said gene into a plant cell and regenerating a plant from the plant cell.

44. A method of producing a useful crop, the method comprises:
cultivating the plant according to claim 32; and
harvesting the plant or a portion thereof.

45. A method of producing a useful crop, the method comprises:
cultivating the plant according to claim 40; and
harvesting the plant or a portion thereof.

46. A breeding marker containing at least a portion of DNA (g) below:
(g) DNA which has a base sequence set forth in SEQ ID NO:3.

47. A breeding agent for modifying yield of a plant or a portion thereof, the breeding agent comprises:
A first DNA encoding any of proteins (a) to (f) below and, upstream of the first DNA, a second DNA (g) below:
(a) a protein which has an amino acid sequence set forth in SEQ ID NO:2;
(b) a protein which has an amino acid sequence having, in the amino acid sequence set forth in SEQ ID NO:2, one or more substituted, deleted, added and/or inserted amino acid, and which has a primary panicle branch number-increasing activity;
(c) a protein which has an amino acid sequence having at least 70% identity with the amino acid sequence set forth in SEQ ID NO:2, and which has a primary panicle branch number-increasing activity;
(d) a protein which is encoded by DNA having the base sequence set forth in SEQ ID NO:1;
(e) a protein which is encoded by DNA that hybridizes under stringent conditions with a strand complementary to a polynucleotide having the base sequence set forth in SEQ ID NO:1, and which has a primary panicle branch number-increasing activity;
(f) a protein which is encoded by DNA having at least 70% identity with the base sequence set forth in SEQ ID NO:1, and which has a primary panicle branch number-increasing activity;
(g) DNA which has a base sequence set forth in SEQ ID NO:3.

48. The plant according to claim 32, the second DNA has a base sequence set forth in SEQ ID NO:5.

49. The plant according to claim 32, the second DNA has a base sequence which maintains bases at the positions 1, 29, 2621, 3474 and 3827 in the sequence set forth in SEQ ID NO:5, respectively and has one or more substituted, deleted, added and/or inserted base, and has an ability to enhance expression of the protein encoded by the first DNA and having a primary panicle branch number-increasing activity.
